SEQUENCE LISTING (8 sequences)

SEQ ID NO: 1 - protein, 4 aa, synthetic construct
  REGION 1..4: note = synthetic sequence; synthetic peptide
RGDS

SEQ ID NO: 2 - protein, 5 aa, synthetic construct
  REGION 1..5: note = synthetic sequence; synthetic peptide
CRGDS

SEQ ID NO: 3 - protein, 6 aa, synthetic construct
  REGION 1..6: note = synthetic sequence; synthetic peptide
CRGDSP

SEQ ID NO: 4 - protein, 5 aa, synthetic construct
  REGION 1..5: note = synthetic sequence; synthetic peptide
PHSRN

SEQ ID NO: 5 - protein, 9 aa, synthetic construct
  REGION 1..9: note = synthetic sequence; synthetic peptide
GWGGRGDSP

SEQ ID NO: 6 - protein, 10 aa, synthetic construct
  REGION 1..10: note = synthetic sequence; synthetic peptide
RGDSPGERCG

SEQ ID NO: 7 - protein, 6 aa, synthetic construct
  REGION 1..6: note = synthetic sequence; synthetic peptide
KGRGDS

SEQ ID NO: 8 - protein, 5 aa, synthetic construct
  REGION 1..5: note = synthetic sequence; synthetic peptide
KRGDS

(19) United States
(12) Patent Application Publication
(10) Pub. No.: US 2024/0050586 A1
(43) Pub. Date: Feb. 15, 2024

(54) COMPOSITIONS AND METHODS FOR MAKING AND USING DOUBLE NETWORK HYDROGELS

(71) Applicant: The Regents of the University of Colorado, a Body Corporate, Denver, CO (US)

(72) Inventors: Alexandra Borelli, Littleton, CO (US); Mark Young, Boulder, CO (US); Balaji V. Sridhar, Denver, CO (US); Venu Akuthota, Lakewood, CO (US); Kristi S. Anseth, Boulder, CO (US)

(21) Appl. No.: 18/361,713

(22) Filed: Jul. 28, 2023

Related U.S. Application Data
(63) Continuation of application No. PCT/US2022/014729, filed on Feb. 1, 2022.
(60) Provisional application No. 63/144,379, filed on Feb. 1, 2021.

(51) Int. Cl. A61K 47/69; C08J 3/075; A61K 47/61; A61K 35/28; A61K 45/06

(52) U.S. Cl. CPC A61K 47/6903; C08J 3/075; A61K 47/61; A61K 35/28; A61K 45/06; C08J 2405/08; C08J 2471/02

(57) ABSTRACT

Embodiments of the instant disclosure relate to novel compositions, combination compositions, methods, and systems for generating and using hydrogels. In certain embodiments, the present disclosure provides for compositions including a polymer backbone having at least one hyaluronic acid backbone functionalized with an aliphatic aldehyde and at least one hyaluronic acid backbone functionalized with a hydrazide and a second polymer backbone including at least one 8-arm poly(ethylene glycol) (PEG). In certain embodiments, the present disclosure provides methods of treating a condition in a subject including administering hydrogels to the subject in the absence or presence of one or more therapeutic agent or cell.

### PRIORITY

This application is a U.S. Continuation application that claims priority to International Application PCT/US2022/014729, filed Feb. 2, 2022, which claims priority to U.S. Provisional Application No. 63/144,379 filed Feb. 1, 2021. These applications are incorporated herein by reference in their entirety for all purposes.

### STATEMENT REGARDING SEQUENCE LISTING

The instant application contains a Sequence Listing which has been submitted via ASCII copy created on Jul. 28, 2023, referred to as ‘106549-764769 CU4987H-US1.xml’ that is 15 kilobytes (KB) in size having 8 sequences and is incorporated herein in its entirety for all purposes.

### FIELD

Embodiments of the instant disclosure generally relate to compositions, methods, and systems for generating hydrogels. Other embodiments disclosed herein generally relate to compositions, methods, and systems for generating and using hydrogels for therapeutic applications in the treatment of health conditions.

### BACKGROUND

Because the general composition and mechanical properties of hydrogels are similar to those of biological tissues, hydrogels are of great interest as biomaterials. Hydrogels can be used in regenerative medicine and can also serve as scaffolding materials or delivery vehicles for small-molecule, protein-based, and cell-based therapies. Viscoelastic hydrogels allow for injection and modulation of their mechanical properties to more closely match that of the native extracellular matrix at the injection site. Viscoelastic hydrogels can be manipulated to recapitulate the in-vivo milieu in part by tuning their elastic behavior through adaptable chemistry. However, substantial degradation and mass loss occurs in viscoelastic hydrogels known to date, which limits their long-term utility. Therefore, there is a need to develop new strategies and formulations for stabilizing viscoelastic hydrogels while maintaining the benefits of the hydrogel adaptable chemistry of use in therapeutic settings.

### SUMMARY

Embodiments of the instant disclosure relate to novel compositions, methods and systems for generating hydrogels (e.g., hybrid network hydrogels). In certain embodiments, the present disclosure provides for compositions including, but not limited to, a first polymer backbone having at least one hyaluronic acid backbone functionalized with an aliphatic aldehyde and at least one hyaluronic acid backbone functionalized with a hydrazide; and, a second polymer backbone having at least one 8-arm poly(ethylene glycol) (PEG), where the combination of the two polymer backbones can produce a hybrid network hydrogel having improved properties.

In some embodiments, the 8-arm PEG of the second polymer backbone can include, but is not limited to, at least one 8-arm PEG functionalized with at least one strained cyclooctyne. In certain embodiments, the 8-arm PEG of the second polymer backbone can include, but is not limited to, at least one 8-arm PEG functionalized with a bicyclononyne or similar functionalizing agent. In accordance with these embodiments, the second polymer backbone including at least one 8-arm PEG can optionally further include, but is not limited to, benzaldehyde-PEG3-azide. In some embodiments, a hyaluronic acid backbone, a PEG macromere, or a combination thereof can be modified with one or more peptide. In some embodiments, the peptide is about 2 to about 50 amino acids in length.

In some embodiments, compositions disclosed herein can include, but are not limited to, an equal or alternatively an unequal concentration of the two polymer backbones. In accordance with these embodiments, one polymer backbone can include at least one hyaluronic acid backbone functionalized with an aliphatic aldehyde and one hyaluronic acid backbone functionalized with a hydrazide compared to the concentration of a second polymer backbone including at least one 8-arm PEG, or less of the polymer backbone having at least one hyaluronic acid backbone functionalized with an aliphatic aldehyde and at least one hyaluronic acid backbone functionalized with a hydrazide compared to the amount of the second polymer backbone having at least one 8-arm PEG. In certain embodiments, compositions disclosed herein can include about 25% by weight of a total crosslink concentration within a polymer backbone including, but not limited to, at least one hyaluronic acid backbone functionalized with an aliphatic aldehyde and at least one hyaluronic acid backbone functionalized with a hydrazide; and about 75% by weight of a total crosslink concentration in a second polymer backbone having at least one 8-arm PEG.

In some embodiments, compositions disclosed herein can further include cells such as stem cells or other therapeutic cell for delivery to a subject. In accordance with these embodiments, stem cells suitable for compositions disclosed herein can include embryonic stem cells, induced pluripotent stem cells, hematopoietic stem cells, neural stem cells, epithelial stem cells, skin stem cells, and mesenchymal stem cells or mesenchymal stromal cells.

In some embodiments, compositions disclosed herein can be formulated in a pharmaceutical composition, which can further include a pharmaceutically acceptable carrier. In accordance with these embodiments, compositions herein can be a hybrid network hydrogel formulated in a pharmaceutical composition, which can further include a pharmaceutically acceptable carrier suitable for injection.

In some embodiments, compositions disclosed herein can further include at least one active agent. In accordance with these embodiments, an active agent included in the composition herein can be released from the hydrogel before, after, or during degradation of the composition. In accordance with these embodiments, the composition can degrade after at least one month.

In some embodiments, the present disclosure provides for methods of forming hydrogels as disclosed herein. In accordance with these embodiments, a method of forming a hydrogel can include combining one polymer backbone having at least one hyaluronic acid backbone functionalized with an aliphatic aldehyde and at least one hyaluronic acid backbone functionalized with a hydrazide with a second polymer backbone having at least one 8-arm poly(ethylene glycol) (PEG), wherein the method does not require external stimulation for hydrogel formation.

In some embodiments, combination compositions disclosed herein can lead to an increase in stress relaxation of the hydrogel by combining increasing concentration of a second polymer backbone having at least one 8-arm PEG to decreasing concentration of the polymer backbone having at least one hyaluronic acid backbone functionalized with an aliphatic aldehyde and at least one hyaluronic acid backbone functionalized with a hydrazide. In some embodiments, combination compositions disclosed herein can lead to a decrease in stress relaxation of certain hydrogel formulations by combining decreasing concentrations of a second polymer backbone having at least one 8-arm PEG to increasing concentrations of the polymer backbone having at least one hyaluronic acid backbone functionalized with an aliphatic aldehyde and at least one hyaluronic acid backbone functionalized with a hydrazide. In accordance with these embodiments, combination compositions and methods of decreasing stress relaxation of a hybrid network hydrogel formulation disclosed herein can lead to an increase in migration of at least one stem cell included in the decreasing or decreased stress relaxation hydrogel composition compared to combination hydrogel formulations not having decreased stress relaxation. In accordance with these embodiments, combination compositions and methods of using decreasing stress relaxation of a hybrid network hydrogel formulation disclosed herein can lead to an increase in secretion of at least one anti-inflammatory cytokine from stem cells included in the decreasing or decreased stress relaxation hydrogel combination composition compared to combination hydrogel formulations not having decreased stress relaxation. In accordance with these embodiments, combination compositions and methods of using increasing stress relaxation of a hybrid network hydrogel formulation disclosed herein can cause an increase in secretion of at least one pro-inflammatory cytokine from stem cells included in a hybrid network hydrogel composition compared to combination hydrogel formulations not having increased stress relaxation.

In other embodiments, hybrid network hydrogels generated using compositions, combination compositions and methods disclosed herein can be used for treating, reducing progression, reducing onset and/or preventing a health condition or disease in a subject in need thereof. In certain embodiments, methods of treating and/or preventing conditions as disclosed herein can include an inflammatory condition, an autoimmune condition, a vascular condition, an orthopedic condition, cancer, or a combination thereof. In accordance with these embodiments, hybrid network hydrogels disclosed herein can be administered to the subject by injection directly or indirectly with respect to the affected tissue or region of the subject. In accordance with these embodiments, hybrid network hydrogels disclosed herein can be administered to the subject by injection through a syringe, a catheter, a trocar, a cannula, or a combination thereof. In certain embodiments, a large gauge needle or catheter can be used to introduce the hybrid network hydrogels of the instant disclosure for acute or prolonged treatment.

### BRIEF DESCRIPTION OF THE DRAWINGS

FIG. 1A is a schematic drawing illustrating macromers used in the formation of hydrogels in accordance with certain embodiments of the present disclosure.

FIG. 1B is a schematic drawing illustrating a viscoelastic HA-hydrazone gel system in accordance with some embodiments of the present disclosure.

FIG. 1C is a schematic drawing illustrating a PEG-triazole gel system mixed with a HA-hydrazone gel system in accordance with certain embodiments of the present disclosure.

FIG. 1D is a schematic drawing illustrating a hydrazone bond formed between the hydrazide and aldehyde groups functionalized on the hyaluronic acid backbone of HA-Hyd and HA-Ald and a bond formed between the (1R,8S,9s)-Bicyclo[6.1.0]non-4-yn-9-ylmethyl (BCN) and benzaldehyde-Peg3-azide (Azide) groups in accordance with some embodiments of the present disclosure.

FIG. 1E is a schematic drawing illustrating an elastic PEG-HA dual network gel having HA-hydrazide, PEG-BCN, and benzaldehyde-PEG3-azide to form stable hydrazone and triazole linkages in accordance with some embodiments of the present disclosure.

FIGS. 2A-2C illustrate examples of dose responses and time courses of gel formation for PEG-HA dual network gels having different ratios of HA-hydrazone to PEG-triazole in accordance with certain embodiments of the present disclosure. FIG. 2A illustrates hydrogel in-still formation. FIG. 2B illustrates swollen modulus of the hydrogels and FIG. 2C illustrates an in-situ modulus summary of the hydrogels.

FIGS. 2D-2J illustrate the effect of viscoelastic properties on hydrogel stress relaxation in accordance with certain embodiments of the present disclosure. FIG. 2D illustrates stress relaxation behavior of the hydrogels. FIG. 2E illustrates average relaxation times calculated as a function of the percentage of hydrazone crosslinks (viscoelasticity) in the hydrogel. FIG. 2F illustrates the percentage of stress relaxed in the hydrogels. FIG. 2G illustrates molar ratios of HA:PEG and Ha-ald:PEG-pHAld. FIG. 2H illustrates the fit stretching parameters (β). FIG. 2I illustrates the average time constant for stress relaxation <τ>. FIG. 2J illustrates a final modulus of hydrogels 24 hours after swelling in PBS.

FIGS. 3A-3C illustrate examples of dose responses and time courses of stress relaxation for PEG-HA dual network gels having different ratios of HA-hydrazone to PEG-triazole in accordance with embodiments of the present disclosure.

FIGS. 4A-4F illustrate examples of time lapse images of a representative PEG-HA dual network gel having 75% elasticity (75% PEG-triazole and 25% HA-hydrazone) ejected from a syringe in accordance with certain embodiments of the present disclosure.

FIGS. 5A-5D illustrate examples of representative images of mesenchymal stem cells (MSCs) that were encapsulated in PEG-HA dual network gels having either 100% elasticity or 12% elasticity 0 days or 4 days after encapsulation in accordance with some embodiments of the present disclosure.

FIG. 5E illustrates an example of mesenchymal stem cells (MSC) morphology characterized as a function of stress relaxation 4 days after encapsulation in a PEG-HA dual network gel in accordance with certain embodiments of the present disclosure.

FIGS. 6A-6G illustrate examples of morphological changes as a function of stress relaxation of MSCs encapsulated in hydrogels having increasing viscoelasticity in accordance with some embodiments of the present disclosure.

FIG. 611 illustrates a representative image illustrating the formation of small clusters in the 88% adaptable hydrazone bond condition in accordance with some embodiments of the present disclosure.

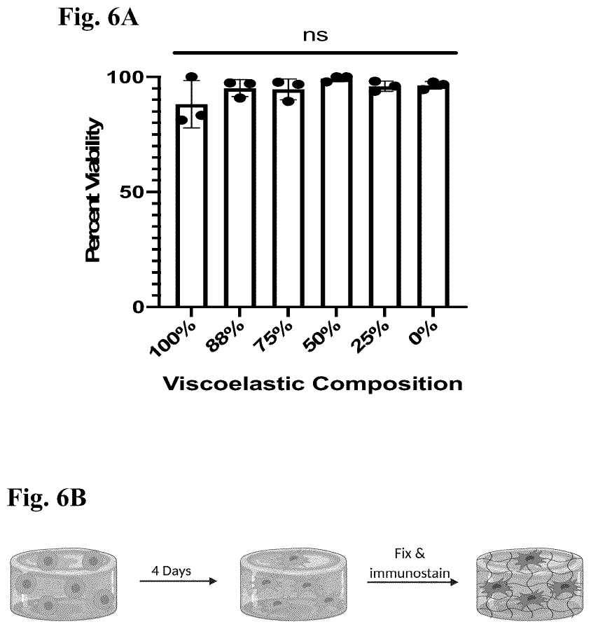

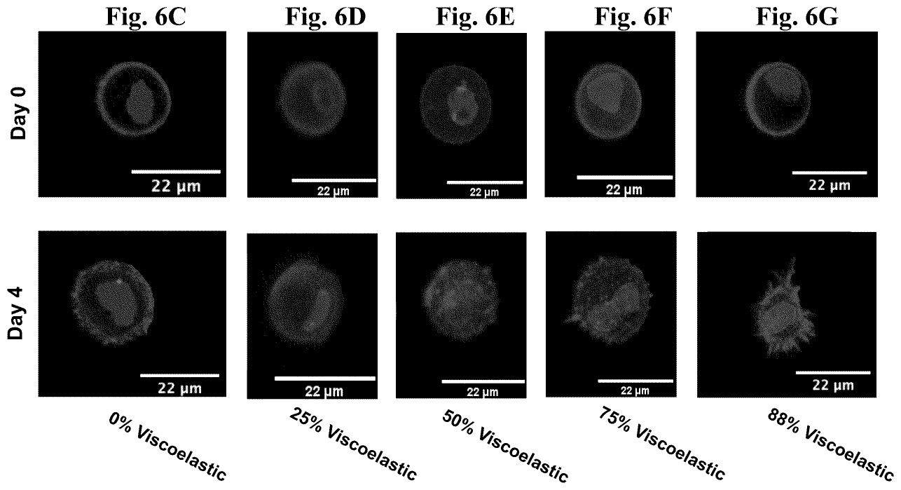

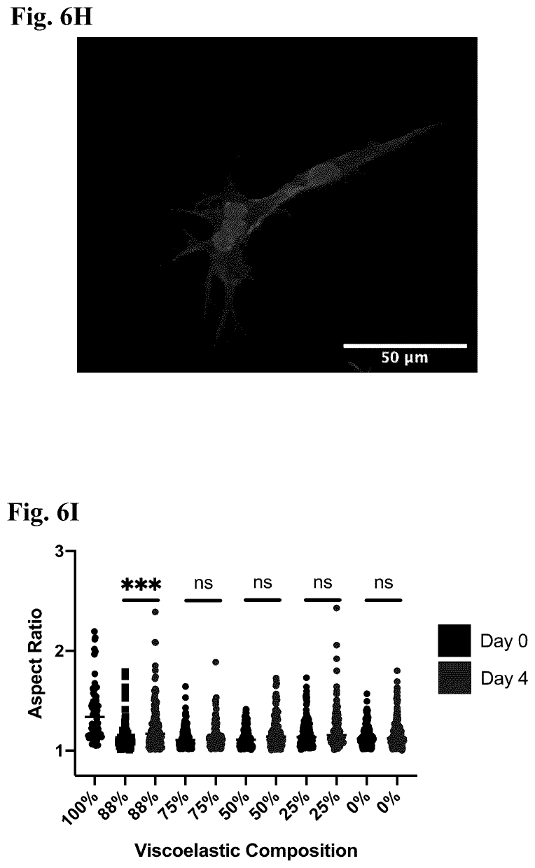

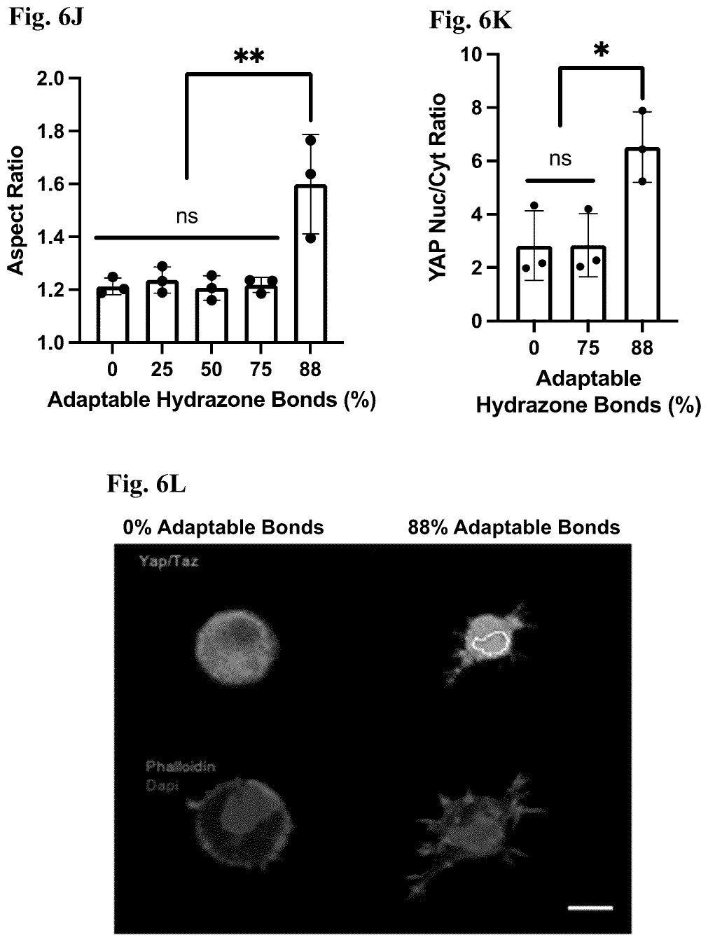

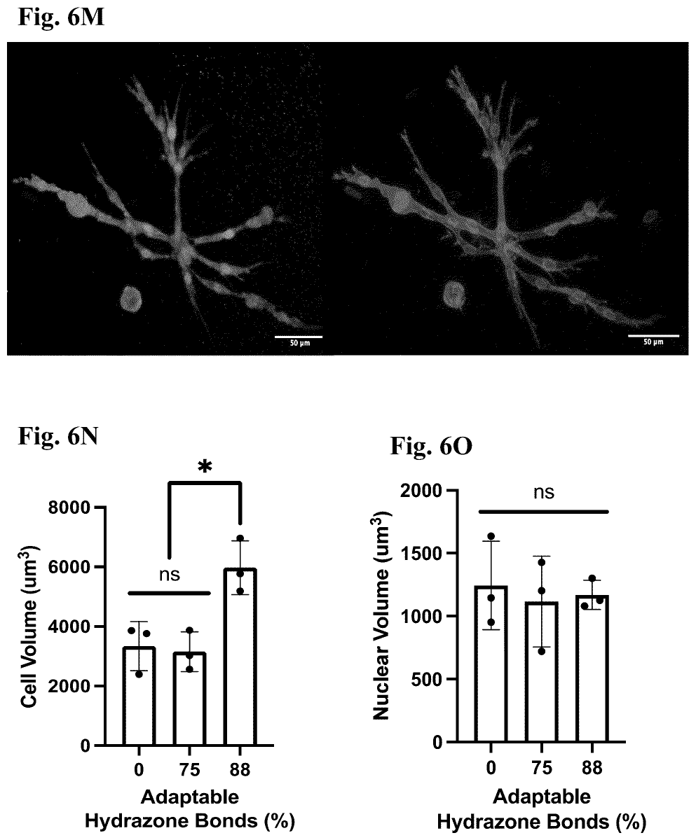

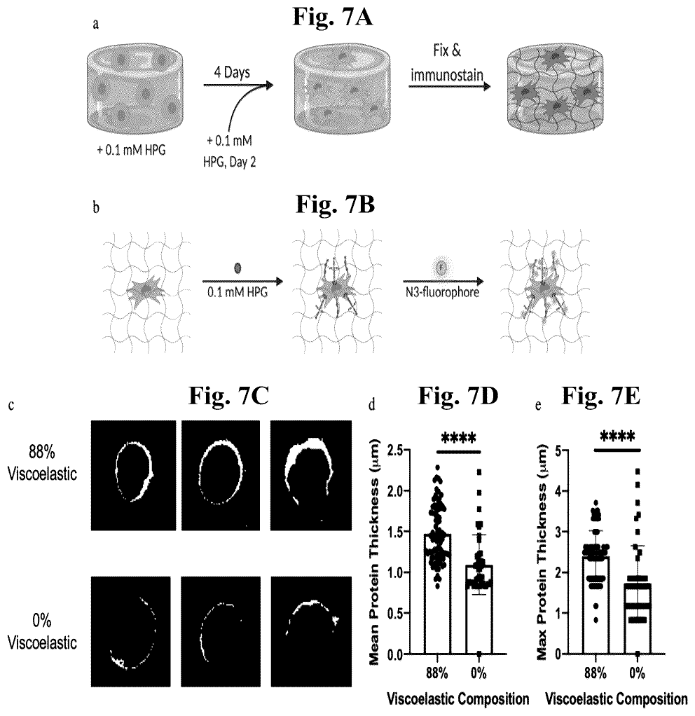

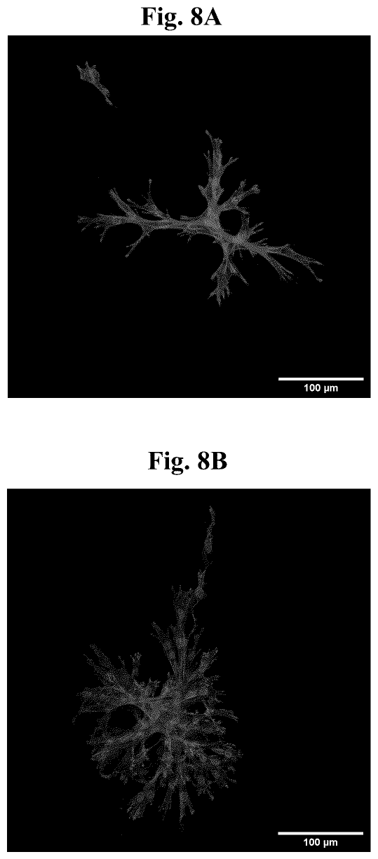

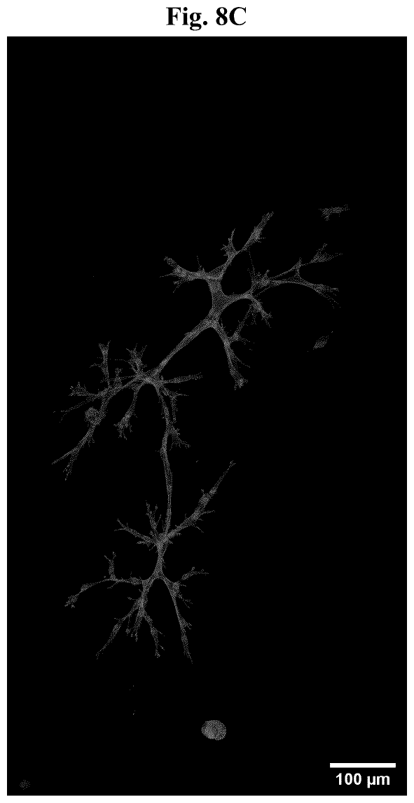

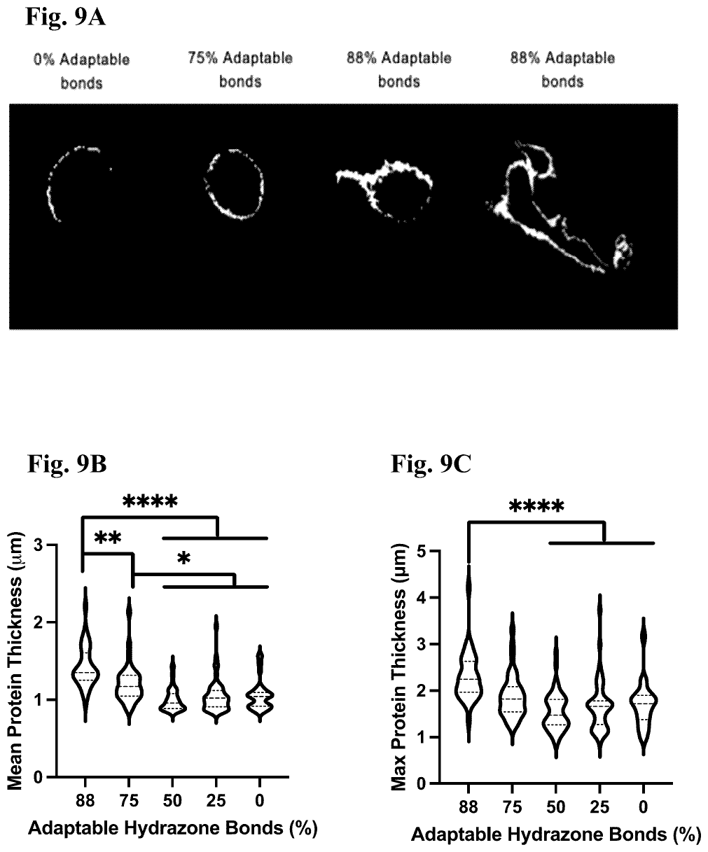

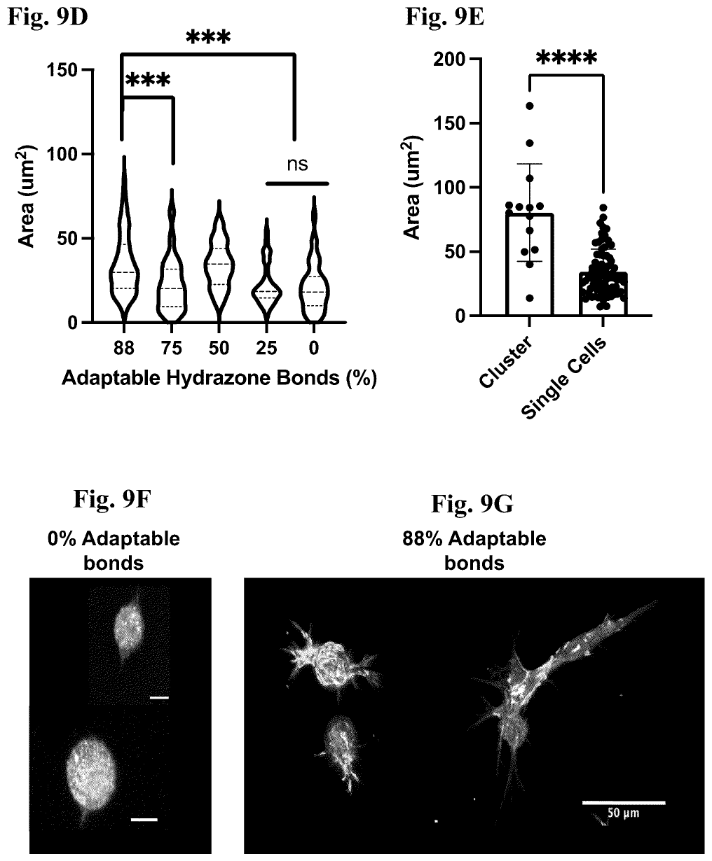

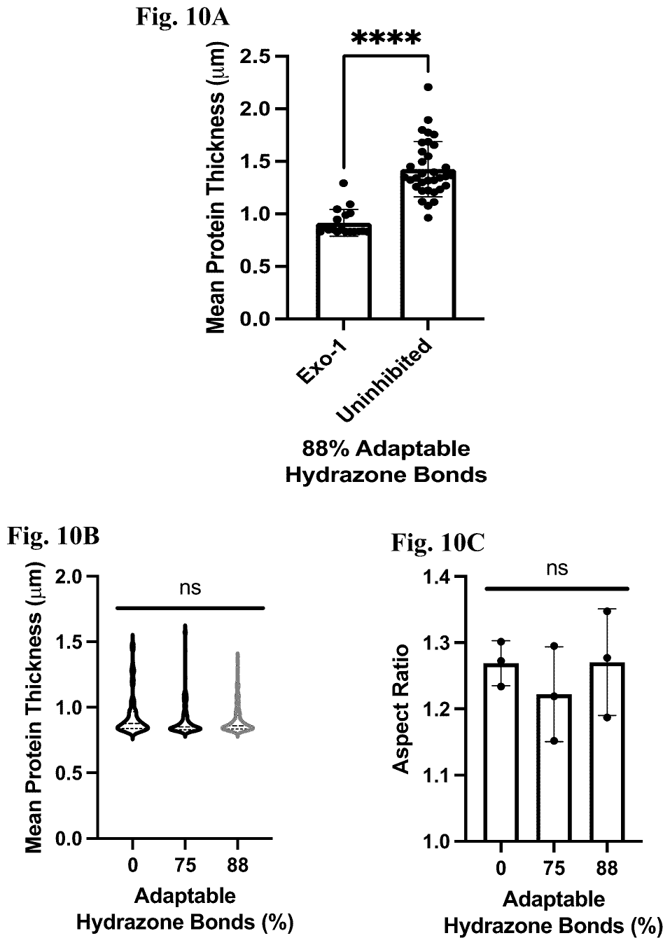

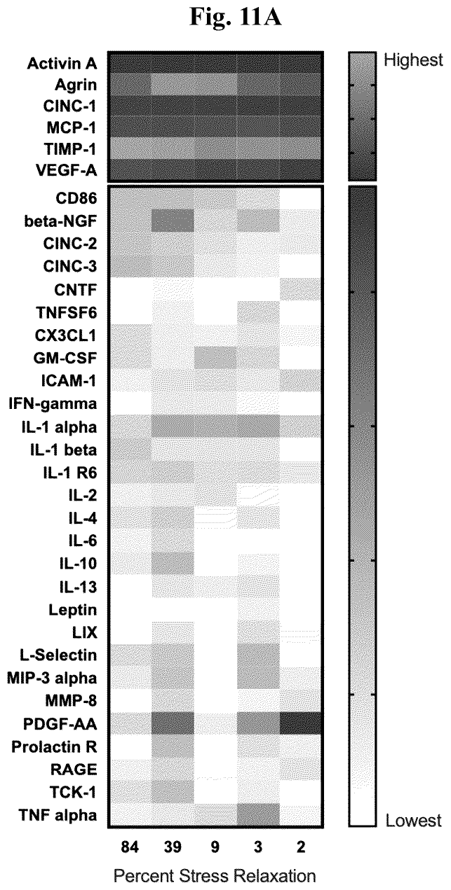

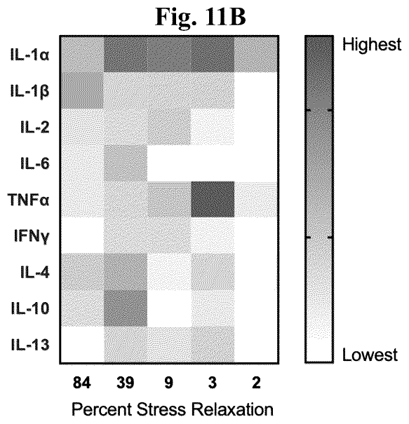

FIGS. 61-6J illustrate morphological changes as a function of stress relaxation in hydrogels in accordance with some embodiments of the present disclosure.

FIG. 6K-6M illustrate Yap/Taz analysis of mesenchymal stem cells after 4 days post-encapsulation in hydrogels in hydrogels in accordance with some embodiments of the present disclosure. FIG. 6K illustrates Yap/Taz nuclearization in the cells. FIG. 6L illustrates (Top) a representative image of a cell shape on Day 0 post-encapsulation in the negative control, 0% viscoelastic condition and (Bottom) a representative image of a cell shape on Day 4 in the negative control. FIG. 6M illustrates (left) a representative image of a Yap/Taz cell signal of a small cluster on Day 4 post-encapsulation, and (right) a representative image of a cell shape on Day 4 of a small cluster illustrating significant cell spreading.

FIG. 6N-6O illustrate the average cellular volume of rMSCs (FIG. 6N) and average nuclear volume (FIG. 6O) after 4 days in the adaptable hydrazone bond conditions in accordance with some embodiments of the present disclosure.

FIGS. 7A-7E illustrate examples of effects of stress relaxation on nascent protein deposition by rMSCs days after encapsulation in a dual network gel in accordance with some embodiments of the present disclosure.

FIGS. 8A-8C illustrate examples of induced migration of MSC cells to form multinuclear structures in hydrogels having high viscoelasticity in accordance with some embodiments of the present disclosure.

FIGS. 9A-9G illustrate the effect of stress relaxation on nascent protein deposition by rMSCs in hydrogels in accordance with some embodiments of the present disclosure. FIG. 9A illustrates representative images of single cell and clustered nascent protein deposition from several viscoelastic conditions. FIG. 9B illustrates a graphical representation of the mean secreted protein thickness. FIG. 9C illustrates the maximum secreted protein. FIG. 9D illustrates the average area of the deposited proteins surrounding the rMSCs in each condition. FIG. 9E illustrates the average area of deposited protein between rMSCs clustered vs single cell rMSCs in the 88% viscoelastic condition. FIG. 9F-9G show representative images depicting deposition of fibronectin in the 0% viscoelastic condition (FIG. 9F) and the 88% viscoelastic condition (FIG. 9G).

FIGS. 10A-10C illustrate the effect of Exo-1 inhibition on nascent protein deposition by rMSCs in hydrogels in accordance with some embodiments of the present disclosure.

FIGS. 11A-11B illustrate examples of cytokine array assays of cytokine secretion of pro- and anti-inflammatory cytokines as a function of hydrogel stress relaxation 4 days after encapsulation in a PEG-HA dual network gel in accordance with some embodiments of the present disclosure.

### DEFINITIONS

Terms, unless specifically defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject comprises a human. In other embodiments, the subject comprises a human in need of bone repair or bone formation.

As used herein, “treatment,” “therapy” and/or “therapy regimen” refer to the clinical intervention made in response to a disease, disorder or physiological condition manifested by a patient or to which a patient can be susceptible. The aim of treatment includes the alleviation or prevention of symptoms, slowing or stopping the progression or worsening of a disease, disorder, or condition and/or the remission of the disease, disorder or condition.

As used herein, “prevent” or “prevention” refers to eliminating or delaying the onset of a particular disease, disorder or physiological condition, or to the reduction of the degree of severity of a particular disease, disorder or physiological condition, relative to the time and/or degree of onset or severity in the absence of intervention.

The term “effective amount” or “therapeutically effective amount” refers to an amount sufficient to effect beneficial or desirable biological and/or clinical results.

“Hydrogel” can refer to an at least partially hydrophilic substance having characterized by high water absorbency. In some embodiments, hydrogel can have an at least partially hydrophilic polymer, superabsorbent polymer or biomacromolecule, for example in a network configuration. In some embodiments, for example, hydrogels can absorb water greater than or equal to about 10 times the hydrogel weight, greater than or equal to about 50 times the hydrogel weight or, greater than or equal to about 100 times the hydrogel weight or more. The term “hydrogel-forming agent”, also termed herein as “hydrogel precursor”, can refer to any chemical compound that can be used to make a hydrogel disclosed herein.

In certain embodiments, the terms “elasticity,” “viscoelasticity,” “degree of stress relaxation,” “stress relaxation,” and/or “relaxation” can be used interchangeably to describe compositions and/or features or states of compositions disclosed herein.

In certain embodiments, “a first polymer network” can be referred to as “a first polymer backbone.” In certain embodiments, “a second polymer network” can be referred to as “a second polymer backbone.” in certain embodiments, “a hydrogel” can be referred to as “a hybrid network hydrogel” and vice versa.

Unless otherwise defined, all technical terms used herein have the same meaning as commonly understood by one of ordinary skill in the art to which this disclosure belongs.

### DETAILED DESCRIPTION OF THE INVENTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

Embodiments of the instant disclosure relate to novel compositions, methods, and systems for generating and using hydrogels (e.g., hybrid network hydrogels) in the treatment of health conditions disclosed herein. In some embodiments, a hydrogel can be formed by using at least one, or more than one type of hydrogel-forming agent; and setting or solidifying the one or more types of hydrogel-forming agent in an aqueous medium to form a three-dimensional network. In certain embodiments, formation of the three-dimensional network can cause the one or more types of hydrogel-forming agent to gel forming hydrogel complexes of use in compositions and methods disclosed herein.

In some embodiments, a hydrogel disclosed herein can include at least one polymeric material. In accordance with these embodiments, the polymeric material can be, a natural polymer material, a synthetic polymer material and combinations thereof. In other embodiments, a polymer suitable for use in compositions, combination compositions and methods disclosed herein can be homopolymeric and/or heteropolymeric. In accordance with these embodiments, a polymer can include, but is not limited to, cross-polymers or co-polymers of any co-monomer distribution, and can be linear, branched, hyperbranched, dendrimeric, or crosslinked to any extent.

In certain embodiments, suitable polymers can include, but are not limited to, gelatin, methylcellulose, hydroxyethyl methyl cellulose, hydroxypropyl methyl cellulose, polyethylene oxide, polyacrylamides, polyacrylic acid, polymethacrylic acid, salts of polyacrylic acid, salts of polymethacrylic acid, poly(2-hydroxyethyl methacrylate), polylactic acid, polyglycolic acid, polyvinylalcohol, polyanhydrides such as poly(methacrylic) anhydride, poly(acrylic) anhydride, polysebasic anhydride, collagen, poly(hyaluronic acid), hyaluronic acid-containing polymers and copolymers, polypeptides, dextran, dextran sulfate, chitosan, chitin, agarose gels, fibrin gels, and combinations thereof. In other embodiments, a polymer suitable for use in composition, combination compositions and methods disclosed herein can be a hydrophilic polymer. In certain embodiments, a hydrophilic polymer can include, but is not limited to, poly(ethylene glycol), polyoxazoline, polyaliphatic polyurethanes, polyether polyurethanes, polyester polyurethanes, polyethylene copolymers, polyamides, polyvinyl alcohols, poly(ethylene oxide), polypropylene oxide, polypropylene glycol, polytetramethylene oxide, polyvinyl pyrrolidone, polyacrylamide, poly(hydroxy ethyl acrylate), poly(hydroxyethyl methacrylate), or mixtures or co-polymers thereof.

In certain embodiments, a hydrogel disclosed herein can contain or further contain in addition to the polymers referenced above, a synthetic polymer poly(ethylene glycol) (PEG). In some embodiments, the PEG for use in compositions, combination compositions and methods disclosed herein can be a PEG-core dendrimer, a PEG block copolymer, or a multi-arm PEG. In certain embodiments, multi-arm PEGs for use in compositions, combination compositions and methods disclosed herein can be 3-arm PEGs, 4-arm PEGS, 6-arm PEGS, or 8-arm PEGs. In other embodiments, a hydrogel disclosed herein can contain functionalized multi-arm PEG. In some embodiments, a functionalized multi-arm PEG can include a multi-arm PEG that has been chemically modified to introduce one or more pendant functional groups into the molecule. In other embodiments, a hydrogel disclosed herein can contain a multi-arm PEG functionalized with a strained cyclooctyne. In accordance with these embodiments, non-limiting examples of a strained cyclooctyne can include, but is not limited to, an aza-dibenzocyclooctyne (ADIBO), bicyclononyne (BCN), dib enzocyclooctyne (DIBO), (OCT), aryl-less octyne (ALO), monofluorinated cyclooctyne (MOFO), difluorinated cyclooctyne (DIFO), biarylazacyclooctynone (BARAC), or a dimethoxyazacyclooctyne (DIMAC) moiety. In some embodiments, a hydrogel disclosed herein can contain a multi-arm PEG functionalized with bicyclononyne ((1R,8S,9s)-Bicyclo[6.1.0]non-4-yn-9-ylmethyl (BCN)). In some embodiments, a hydrogel composition or combination composition disclosed herein can include an 8-arm PEG functionalized with bicyclononyne.

In certain embodiments, a hydrogel disclosed herein can contain hyaluronic acid (HA). HA is a non-sulphated glycosaminoglycan (GAG) in the extracellular matrix (ECM) of many soft connective tissues, composed of alternating units of D-glucuronic acid and N-acetyl-D-glucosamine with a molecular weight (MW) up to about 6 MDa, linked together via alternating β-1,4 and β-1,3 glycosidic bonds. In some embodiments, HA can be extracted from natural tissues including, but not limited to, the connective tissue of vertebrates, from the human umbilical cord and from cocks' combs. In other embodiments, HA can be prepared by microbiological methods, for example, to minimize the potential risk of transferring infectious agents to a subject, and to increase product uniformity, quality, and/or availability. In certain embodiments, a hydrogel disclosed herein can contain functionalized HA. Functionalized HA can include functionalized HA that has been chemically modified. In certain embodiments, functionalized HA can include functionalized HA that has been chemically modified to introduce one or more pendant functional groups into the molecule. In yet other embodiments, a functionalized HA disclosed herein can have at least one aldehyde group functionalized on the HA backbone. In other embodiments, a functionalized HA disclosed herein can have at least one hydrazide group functionalized on the HA backbone. In other embodiments, a functionalized HA disclosed herein can have more than one group of agents on the HA backbone.

In some embodiments, a HA backbone, a PEG macromer, or a combination thereof disclosed herein can be further modified with at least one peptide. In accordance with these embodiments, a peptide can include a peptide length of about 2 amino acids to about 50 amino acids; about 2 amino acids to about 40 amino acids or about 2 amino acids to about 30 amino acids or less than 10 amino acids. In other embodiments, a HA backbone, a PEG macromer, or a combination thereof can be modified to further include a cell adhesion peptide. As used herein, a “cell adhesion peptide” can refer to an amino acid sequence obtained from an adhesion protein to which cells bind via a receptor-ligand interaction. In accordance with these embodiments, varying the cell adhesion peptide and concentrations thereof in the solution can allow for the ability to control the stability of the cellular attachment to the resulting hydrogel composition. In some embodiments, a cell adhesion peptide can have a final hydrogel concentration of about 1 μM to about 10 mM. In some embodiments, a cell adhesion peptide can have a final hydrogel concentration of about 1 μM, or about 50 μM, or about 100 μM, or about 500 μM, or about 1 mM, or about 2 mM, or about 5 mM, or up to about 10 mM or any concentration within the ranges disclosed herein.

In some embodiments, a “cell adhesion peptide” can include a peptide ligand for integrin binding. Suitable peptide ligands for integrin binding include, for example, RGD, RGDS (SEQ ID NO: 1), CRGDS (SEQ ID NO: 2), CRGDSP (SEQ ID NO: 3), PHSRN (SEQ ID NO: 4), GWGGRGDSP (SEQ ID NO: 5), RGDSPGERCG (SEQ ID NO: 6), KRGDS (SEQ ID NO: 8). In some embodiments, a peptide ligand for integrin binding used herein can be a benzaldehyde functionalized RGD peptide. In some embodiments, a peptide ligand for integrin binding disclosed of use herein can be benzaldehyde-KGRGDS (SEQ ID NO: 7). In some embodiments, a peptide ligand for integrin binding can have a final hydrogel concentration of about 1 μM to about 10 mM, or about 500 μM to about 5 mM, or about 1 mM to about 2 mM.

In some embodiments, hydrogels disclosed herein can include a hybrid network hydrogel. In accordance with these embodiments, a hybrid network hydrogel includes hydrogels formed from two polymer backbones for example, forming a single hydrogel complex. In certain embodiments, a hydrogel disclosed herein can contain at least two polymer backbones. In accordance with these embodiments, two polymer backbones can include, but is not limited to, a hydrogel where each polymer backbone has unique polymer components, properties, features and/or have opposite mechanical properties. In some embodiments, hydrogels disclosed herein can contain a polymer backbone that yields a stiff, brittle network or a polymer backbone that yields a soft, ductile network. In some embodiments, two polymer backbones disclosed herein can use any of the chemical compounds and polymers described herein for use to generate combination polymer-containing hydrogels disclosed herein.

In some embodiments, a polymer backbone of compositions, combination compositions and uses disclosed herein can include at least one hyaluronic acid backbone. In accordance with these embodiments, a polymer backbone can include, but is not limited to, at least one functionalized hyaluronic acid (HA) backbone. In certain embodiments, a polymer backbone disclosed herein can include, but is not limited to, hydrazide groups functionalized on the HA backbone. In some embodiments, a polymer backbone disclosed herein can include, but is not limited to, aldehyde groups functionalized on the HA backbone. In certain embodiments, a polymer backbone disclosed herein can include, but is not limited to, at least one functionalized HA backbone having aldehyde groups and at least one functionalized HA backbone having hydrazide groups. In certain embodiments, a polymer backbone disclosed herein can include, but is not limited to, a combination of functionalized HA backbones having aldehyde groups and functionalized HA backbones having hydrazide groups where at least one hydrazone bond is formed between the hydrazide and aldehyde groups. In some embodiments, a polymer backbone can include, but is not limited to, hydrazone bonds between hydrazide and aldehyde groups of the functionalized HA backbones forming a viscoelastic HA gel. In certain embodiments, ‘viscoelasticity’ as referenced herein can refer to a property of materials that exhibit both viscous and elastic characteristics when undergoing deformation. In some embodiments, a polymer backbone disclosed herein having hydrazone bonds between hydrazide and aldehyde groups of the functionalized HA backbones can be the polymer backbone that yields a soft, ductile network.

In some embodiments, a polymer backbone in compositions and methods of use disclosed herein can include, but is not limited to, at least one 8-arm PEG. In certain embodiments, a polymer backbone in compositions and methods of use disclosed herein can include, but is not limited to, at least one functionalized 8-arm PEG. In other embodiments, a polymer backbone in compositions and methods of use disclosed herein can include, but is not limited to, at least one 8-arm PEG functionalized with a bicyclononyne (PEG-BCN). In other embodiments, a polymer backbone herein in compositions and methods of use disclosed herein can include, but is not limited to, benzaldehyde-PEG3-azide. In some embodiments, a polymer backbone in compositions and methods of use disclosed herein can include, but is not limited to, a combination of 8-arm PEG functionalized with a bicyclononyne (BCN) and benzaldehyde-PEG3-azide having at least one bond formed between the BCN and azide groups of the benzaldehyde-PEG3-azide. In certain embodiments, a polymer backbone in compositions and methods of use disclosed herein can include, but is not limited to, a polymer backbone having bonds formed between the BCN and azide groups of the benzaldehyde-PEG3-azide yielding a polymer backbone having a stiff, brittle network.

In certain embodiments, a benzaldehyde-PEG3-azide can be first conjugated to a HA-hydrazide. In accordance with these embodiments, a HA-aldehyde and a HA-hydrazide (now containing benzaldehyde-PEG3-azide) can be combined. In some embodiments, a PEG-BCN can then be added to a combination of a HA-aldehyde and a HA-hydrazide, wherein the HA-hydrazide contains benzaldehyde-PEG3-azide.

In some embodiments, a hydrogel disclosed herein can contain at least two polymer backbones. In accordance with these embodiments, a hydrogel disclosed herein can contain at least two polymer backbones having hydrazone bonds between hydrazide and aldehyde groups of the functionalized HA backbones; and a polymer backbone having bonds formed between the PEG-BCN and azide groups of the benzaldehyde-PEG3-azide. In certain embodiments, a hydrogel disclosed herein having these referenced combined polymer backbones can result in formation of a near or essentially irreversible bond between the hydrazide and benzaldehyde when the benzaldehyde-PEG3-azide reacts onto the HA backbone with hydrazide. In some embodiments, a hydrogel disclosed herein can be formed using at least two polymer backbones (e.g., a polymer backbone herein having hydrazone bonds between hydrazide and aldehyde groups of the functionalized HA backbones and a polymer backbone herein having bonds formed between the PEG-BCN and azide groups of the benzaldehyde-PEG3-azide) where gel formation does not require any external stimulation for gel stabilization. In some embodiments, a hydrogel having two polymer backbones disclosed can gel or form a gel in about 2 to about 10 minutes. In accordance with these embodiments, a hydrogel having two polymer backbones disclosed can gel or form a gel in about 2 minutes or less, about 3 minutes or less, about 4 minutes or less, about 5 minutes or less, about 6 minutes or less, about 7 minutes or less, about 8 minutes or less, about 9 minutes or less, or about 10 minutes or less or up to about 30 minutes or less.

In certain embodiments, any hydrogel or hydrogel combination disclosed herein having two polymer backbones disclosed herein can be stable for about 10 days or more, about 15 days or more, about 1 month or more, about 2 months or more, about 3 months or more, about 4 months or more, about 5 months or more or about 6 months. In certain embodiments, any hydrogel or hydrogel combination disclosed herein having two polymer backbones disclosed herein can be stable for about 15 days to about 6 months or more. In some embodiments, a hydrogel having two polymer backbones disclosed herein can degrade over time. In certain embodiments, a hydrogel having two polymer backbones disclosed herein can degrade over about 15 days to about 6 months. In certain embodiments, a hydrogel having two polymer backbones disclosed herein can degrade over about 10 days or more, about 15 days or more, about 1 month or more, about 2 months or more, about 3 months or more, about 4 months or more, about 5 months or more or about 6 months.

In some embodiments, a hydrogel disclosed herein can contain at least two polymer backbones in different ratios. In accordance with these embodiments, a hydrogel disclosed herein can contain at least two polymer backbones in different ratios of about 1:2 or about 1:3 or about 1:4 or other predetermined ratio. In other embodiments, a hydrogel disclosed herein can contain at least two polymer backbones and have a shear storage modulus of about 300 to about 500 Pa; about 350 to about 500 Pa; about 350 to about 475 Pa; or about 350 to about 450 Pa. In some embodiments, a hydrogel disclosed herein can contain at least two polymer backbones and have a shear storage modulus of about 400 Pa.

In other embodiments, a polymer backbone disclosed herein can generate a stiff, brittle network while another polymer backbone disclosed herein can a generate soft, ductile network, the ratio of the two polymer backbones can influence on the properties of the gel to the desired stiffness, pliable or soft network. In some embodiments, a hydrogel disclosed herein can contain two polymer backbones at about a 1:1 ratio, about a 1:2 ratio, about a 1:3 ratio, about a 1:4 ratio, or about a 1:5 ratio of the polymers as selected and disclosed herein.

In some embodiments, a hydrogel disclosed herein can include two polymer backbones where one polymer backbone includes viscoelastic hydrazone bonds between hydrazide and aldehyde groups of the functionalized HA backbones that can be present at about 1% to about 99% or about 1% to about 50% of the total crosslink concentration within the hydrogel. In other embodiments, the other or second polymer backbone can contain elastic bonds formed between the BCN and azide groups of the PEG-BCN and the benzaldehyde-PEG3-azide can be present at about 1% to about 99% or about 1% to about 50% of the total crosslink concentration. In some embodiments, a hydrogel disclosed herein can have two polymer backbones where one polymer backbone contains viscoelastic hydrazone bonds between hydrazide and aldehyde groups of the functionalized HA backbones that can be present at about 10% to about 25% of the total crosslink concentration. In some embodiments, the other or second polymer backbone herein can contain elastic bonds formed between the BCN and azide groups of the PEG-BCN and the benzaldehyde-PEG3-azide that can be present at about 75% to about 90% of the total crosslink concentration. In some embodiments, a hydrogel disclosed herein can have two polymer backbones where one polymer backbone contains viscoelastic hydrazone bonds between hydrazide and aldehyde groups of the functionalized HA backbones that can be present at about 25% of the total crosslink concentration. In some embodiments, the other or second polymer backbone disclosed herein can contain elastic bonds formed between the BCN and azide groups of the PEG-BCN and benzaldehyde-PEG3-azide that can be present at about 75% of the total crosslink concentration.

In certain embodiments, a hydrogel disclosed herein can contain at least 5% alkyl hydrazone bonds, wherein at least about 95% of the functional HA-hydrazide arms can be functionalized with benzaldehyde-PEG-azide. In some embodiments, a hydrogel disclosed herein can contain about 5% to about 100% alkyl hydrazone bonds, where about 95% to about 0% of the functional HA-hydrazide arms can be functionalized with benzaldehyde-PEG-azide. In some embodiments, a hydrogel disclosed herein can contain about 5%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or 100% alkyl hydrazone bonds. In accordance with these embodiments, a hydrogel disclosed herein can contain 100%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or 0% of the functional HA-hydrazide arms can be functionalized with benzaldehyde-PEG-azide. In some embodiments, a hydrogel disclosed herein can contain 100%, about 88%, about 75%, about 50%, about 25%, about 15%, about 5%, or 0% alkyl hydrazone bonds.

In certain embodiments, a hydrogel disclosed herein can include two polymer backbones where increasing concentrations of benzaldehyde-PEG3-azide can increase stress relaxation of the hydrogel in a dose dependent manner. In other embodiments, a hydrogel disclosed herein can have two polymer backbones wherein increasing concentrations of benzaldehyde-PEG3-azide can decrease the stress relaxation of the gel in a dose dependent manner. In some embodiments, a hydrogel disclosed herein can have two polymer backbones wherein increasing concentrations of benzaldehyde-PEG3-azide can increase the time constant for stress relaxation of the gel in a dose dependent manner. In other embodiments, a hydrogel disclosed herein can include two polymer backbones where increasing concentration of benzaldehyde-PEG3-azide can prolong the time until hydrogel formation occurs.

In other embodiments, a hydrogel generated from two polymer backbones disclosed herein can include a Young's modulus of about 0.5 to about 2000 Pa. In some embodiments, a hydrogel generated from the two polymer backbones disclosed herein can include a Young's modulus of about 0.5 Pa to about 2000 Pa; or about 0.5 Pa, about 10 Pa, about 50 Pa, about 100 Pa, about 200 Pa, about 300 Pa, about 400 Pa, about 500 Pa, about 600 Pa, about 700 Pa, about 800 Pa, about 900 Pa, about 1000 Pa, about 1250 Pa, about 1500 Pa, about 1750, or about 2000 Pa.

In some embodiments, a hydrogel described herein can be porous. In other embodiments, pores can be homogenously dispersed throughout the hydrogel. In some embodiments, pores can be heterogeneously dispersed throughout the hydrogel. In yet other embodiments, hydrogels described herein can include micron-sized pores. In accordance with these embodiments, hydrogels described herein can include pores having an approximate diameter of about 0.1 μm to about 900 μm, about 1 μm to about 500 μm, or about 10 μm to about 250 μm or about 100 μm to about 200 μm.

In certain embodiments, hydrogels including, but not limited to, complex hydrogels or combination hydrogels disclosed herein can be formulated into a pharmaceutical composition. In accordance with these embodiments, pharmaceutical compositions containing one or more hydrogel or hydrogel complex disclosed herein can further includes a pharmaceutically acceptable carrier, diluent or excipient. Any of the pharmaceutical compositions to be used in the present methods can include pharmaceutically acceptable carriers, excipients, or stabilizers in the form of lyophilized formations or aqueous solutions.

The carrier in the pharmaceutical composition must be “acceptable” in the sense that it is compatible with the active ingredient of the composition, and preferably, capable of stabilizing the active ingredient and not deleterious to the subject to be treated. For example, “pharmaceutically acceptable” can refer to molecular entities and other components of compositions including those that are physiologically tolerable and do not typically produce untoward reactions when administered to a mammal (e.g., a human). In some examples, the “pharmaceutically acceptable” carrier used in the pharmaceutical compositions disclosed herein can be those approved by a regulatory agency of the Federal or a state government or listed in the U.S. Pharmacopeia or other generally recognized pharmacopeia for use in mammals, and more particularly in humans.

Pharmaceutically acceptable carriers, including buffers, are well known in the art, and can include phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

In some embodiments, the pharmaceutical compositions or formulations disclosed herein can be for parenteral administration, such as intravenous, intra-articular injection, intracerebroventricular injection, intra-cisterna magna injection, intra-parenchymal injection, or a combination thereof. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Pharmaceutical compositions disclosed herein can further include additional ingredients, for example preservatives, buffers, tonicity agents, antioxidants and stabilizers, nonionic wetting or clarifying agents, viscosity-increasing agents, and the like. The pharmaceutical compositions disclosed herein can be packaged in single unit dosages or in multidosage forms.

Formulations suitable for parenteral administration disclosed herein can include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents. Aqueous solutions can be suitably buffered (preferably to a pH of from 3 to 9). The preparation of suitable parenteral formulations under sterile conditions is readily accomplished by standard pharmaceutical techniques well known to those skilled in the art.

The pharmaceutical compositions to be used for in vivo administration should be sterile. This is readily accomplished by, for example, filtration through sterile filtration membranes. Sterile injectable solutions are generally prepared by incorporating hydrogels in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

In certain embodiments, pharmaceutical compositions disclosed herein can also include other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycols.

In certain embodiments, any of the compositions (e.g., hydrogels and hybrid network hydrogels) described herein can be used for scaffolding materials or delivery vehicles for small-molecule, protein-based, antibody-based, anti-microbial-based and cell-based therapies. In certain embodiments, hydrogels described herein can include stem cells. Non-limiting examples of stem cells suitable for use herein can include embryonic stem cells, induced pluripotent stem cells, hematopoietic stem cells, neural stem cells, epithelial stem cells, skin stem cells, and mesenchymal stem cells or mesenchymal stromal cells. In certain embodiments, hydrogels described herein can include mesenchymal stem cells (MSCs). In other embodiments, hydrogels described herein can include adipose tissue stem cells, chondrocyte tissue stem cells, osteocyte tissue stem cells, or the combination thereof. Stem cells suitable for use herein can be obtained from any source known in the art. In certain embodiments, stem cells can be obtained from embryonic or adult tissue including, but not limited to endothelial tissue from umbilical cord vein, endothelial tissue from foreskin, endometrial tissue, human embryonic stem cells, dental, skin and adipose tissue or other tissue. Pluripotent cells can also be artificially produced by inducing pluripotency.

In certain embodiments, hydrogels disclosed herein can include stem cells dispersed through the hydrogel either homogenously or heterogeneously having concentrated pockets of cells or as desired. In other embodiments, stem cells can be encapsulated by the hydrogels described herein. In some embodiments, hydrogels described herein can include about 0.1 million stem cells/mL to about 40 million stem cells/mL, about 0.5 million stem cells/mL to about 30 million stem cells/mL, about 1 million stem cells/mL to about 20 million stem cells/mL, about 1.5 million stem cells/mL to about 10 million stem cells/mL, or about 2 million stem cells/mL to about 2 million stem cells/mL.

In some embodiments, the ratio of the two polymer backbones includes a hydrogel disclosed herein that can be modulated to influence one or more cellular functions, including but not limited to, migration, spreading, proliferation, and differentiation. In certain embodiments, a hydrogel disclosed herein having at least one stem cell can have two polymer backbones wherein decreasing concentration of benzaldehyde-PEG3-azide can increase stem cell spreading in a dose dependent manner. In certain embodiments, a hydrogel disclosed herein housing or having at least one stem cell can include two or more polymer backbones wherein the ratio of the two or more polymer backbones can be modulated or adjusted to induce or stimulate migration of the at least one stem cell to form at least one multinuclear structure.

In certain embodiments, hydrogels disclosed herein can include additional components for delivery to a subject in need thereof. In accordance with these embodiments, hydrogels disclosed herein can further include cells. In accordance with these embodiments, hydrogels disclosed herein can further include cells of the same or different origin of the subject to be treated. In other embodiments, hydrogels disclosed herein can further include cells of the derived from the subject to be treated, related or unrelated subject. In other embodiments, hydrogels disclosed herein can further include cells that deposit extracellular matrix. In some embodiments, hydrogels disclosed herein can include cells having nascent protein deposition. In some embodiments, hydrogels disclosed herein can include cells having nascent protein deposition that can contribute to cell spreading, expansion and/or survival. In some embodiments, hydrogels disclosed herein can include exosomes.

In certain embodiments, hydrogels disclosed herein can include stem cells of use for one or more health conditions; for example, for delivery to a subject to treat a health condition. In accordance with these embodiments, stem cells for use herein can include hematopoietic stem cells (HSCs), mesenchymal stem cells (MSCs), endothelial progenitor cells (EPCs), or any combination thereof. In some embodiments, stem cells for use herein can be isolated from bone marrow. In some embodiments, stem cells for use herein can be isolated from peripheral blood. In some embodiments, stem cells for use herein can be isolated from umbilical cord blood. In some embodiments, stem cells for use herein can be isolated from amniotic tissue. In some embodiments, stem cells for use herein can be isolated from peripheral blood. In some embodiments, stem cells for use herein can be isolated from adipose tissue. In some embodiments, stem cells for use herein can be isolated from autologous peripheral blood, umbilical cord blood, amniotic tissue, adipose tissue, teeth, and/or bone marrow. As used herein, the term “autologous” refers to peripheral blood, umbilical cord blood, amniotic tissue, adipose tissue, and/or bone marrow obtained from the same subject to be treated with the hydrogels disclosed herein. In some other embodiments, stem cells for use herein can be isolated from allogeneic peripheral blood, umbilical cord blood, amniotic tissue, adipose tissue, and/or bone marrow. As used herein, the term “allogeneic” refers to peripheral blood, umbilical cord blood, amniotic tissue, adipose tissue, and/or bone marrow obtained from a different subject of the same species as the subject to be treated with the hydrogels disclosed herein.

In some embodiments, hydrogels disclosed herein can include cells having nascent protein deposition that can contribute to cell secretion of protein. In accordance with these embodiments, hydrogels disclosed herein having encapsulated cells can secrete about 0.5 μm to about 5.0 μm of protein. In some embodiments, hydrogels disclosed herein having encapsulated cells therein can secrete about 0.5 μm to about 5.0 μm, or 0.5 μm, about 1.0 μm, about 1.5 μm, about 2.0 μm, about 2.5 μm, about 3.0 μm, about 3.5 μm, about 4.0 μm, about 4.5 μm, or about 5.0 μm of protein. In certain embodiments, proteins can include fibronectin or other ECM related protein.

In certain embodiments, hydrogels disclosed herein having encapsulated cells therein can include a protein thickness of about 0.5 μm to about 5.0 μm. In some embodiments, hydrogels disclosed herein having cells encapsulated therein can have a protein thickness of about 0.5 μm, about 1.0 μm, about 1.5 μm, about 2.0 μm, about 2.5 μm, about 3.0 μm, about 3.5 μm, about 4.0 μm, about 4.5 μm, or about 5.0 μm.

In certain embodiments, hydrogels disclosed herein having encapsulated cells therein can include a heterogeneous distribution of protein within the cells. In some embodiments, hydrogels disclosed herein having encapsulated cells therein can include protein localized to one or more spreading arms of the cell. In some embodiments, hydrogels disclosed herein having encapsulated cells therein can have protein localized to junctions where two cells clustered together.

In certain embodiments, hydrogels disclosed herein having encapsulated cells therein can deposit proteins; for example, surrounding the encapsulated cells. In accordance with these embodiments, an average area of the deposited proteins surrounding a cell encapsulated in a hydrogel disclosed herein can be from about 0.5 μm² to about 150 μm². In some embodiments, an average area of the deposited proteins surrounding a cell encapsulated in a hydrogel disclosed herein can be about 0.5 μm², about 1.0 μm², about 2.5 μm², about 5.0 μm², about 7.5 μm 2, about 10 μm², about 12.5 μm², about 15.0 μm², about 17.5 μm², about 20 μm², about 25 μm 2, about 30 μm², about 35 μm², about 40 μm², about 45 μm², about 50 μm², about 55 μm², about 60 μm², about 65 μm², about 70 μm², about 75 μm², about 80 μm², about 85 μm², about 90 μm², about 95 μm², about 100 μm², about 110 μm², about 120 μm², about 130 μm², about 140 μm², or about 150 μm².

In some embodiments, hydrogels of two polymer backbones can further include a hydrogel having at least one stem cell (e.g. mesenchymal stem cell). In accordance with these embodiments, hydrogels of two polymer backbones further including a hydrogel having at least one stem cell can be modulated to influence the secretory profile of the stem cell, for example, modulate the delivery of cytokines or other agents released by the stem cell. In certain embodiments, decreasing the stress relaxation of a hydrogel having at least one stem cell as disclosed herein can upregulate secretion of cytokines from the stem cell. In accordance with these embodiments, cytokines modulated by decreasing the stress relaxation of a hydrogel having at least one stem cell as disclosed herein can include, but is not limited to, Activin A, Agrin, CINC (cytokine-induced neutrophil chemoattractant)-1, MCP-1 (monocyte chemoattractant protein-1), TIMP-1 (Tissue inhibitor matrix metalloproteinase 1), VEGF-A (vascular endothelial growth factor A), CD86 (Cluster of Differentiation 86), beta-NGF (Nerve growth factor), CINC-2, CINC-3, CNTF (Ciliary Neurotrophic Factor), TNFSf6 (tumor necrosis factor (ligand) superfamily, member 6), CX3CL1 (C-X3-C Motif Chemokine Ligand 1), GM-CSF (Granulocyte-macrophage colony-stimulating factor), ICAM-1 (Intercellular Adhesion Molecule 1), INF (interferon)-gamma, IL (interleukin)-1 alpha, IL-1 beta, IL-1 R6, IL-2, IL-4, IL-10, IL-13, leptin, LIX (LPS-induced CXC chemokine), L-selectin, MIP-3 alpha (Macrophage inflammatory protein-3 alpha), MMP-8 (matrix metalloproteinase-8), PDGF-AA (Platelet-Derived Growth Factor AA), prolactin R, RAGE (Receptor for advanced glycosylation end product.), TCK-1 (Thymus chemokine-1) and TNF (tumor necrosis factor)-alpha. In some embodiments, a hydrogel disclosed herein having at least one stem cell can include two polymer backbones where decreasing concentration of benzaldehyde-PEG3-azide can increase secretion of at least one anti-inflammatory cytokine in a dose dependent manner from the stem cell embedded therein. In other embodiments, a hydrogel disclosed herein having at least one stem cell can have two polymer backbones wherein decreasing concentration of benzaldehyde-PEG3-azide can increase IL-10, IL-6, or the combination thereof in a dose dependent manner from the stem cell embedded therein. In some embodiments, a hydrogel disclosed herein having at least one stem cell can include two polymer backbones wherein increasing concentration of benzaldehyde-PEG3-azide can increase secretion of at least one pro-inflammatory cytokine in a dose dependent manner from the stem cell embedded therein. In some embodiments, a hydrogel disclosed herein having at least one stem cell can include two polymer backbones wherein increasing concentration of benzaldehyde-PEG3-azide can increase TNF-alpha in a dose dependent manner from the stem cell embedded therein.

In some embodiments, a hydrogel of use in compositions, combination compositions and methods disclosed herein can further include at least one active agent. In some embodiments, an active agent can include, but is not limited to, any substance or combination of substances, intended to furnish a biological activity or to otherwise have direct effect in the diagnosis, cure, mitigation, treatment, reduced progression of, or prevention of disease, or to have direct effect in restoring, correcting or modifying physiological functions in a subject. In certain embodiments, an active agent for uses disclosed herein can be small molecule, a peptide, a polynucleotide, a genetically modified cell or an antibody, an antibody fragment or a combination thereof. In other embodiments, a hydrogel disclosed herein can include an active agent or can release an active agent at the time of injection, immediately after injection, at a localized region of administration, at a targeted region of the subject to treat a condition, at a constant rate for the duration of the degradation of the hydrogel after injection, once the hydrogel completely degrades after injection, or a combination thereof.

In certain embodiments, any of the compositions (e.g., hydrogels) described herein can be used for treating, reducing onset, reducing progression, or preventing or both treating and preventing a health condition or for elective surgery or treatment in a subject. In some embodiments, hydrogels disclosed herein can treat, reduce onset, reduce progression or prevent a condition including, but not limited to, an inflammatory condition or disease, an autoimmune condition or disease, a vascular condition or disease, an orthopedic condition, cancer, a spinal or brain injury (traumatic brain injury), of use in tissue or organ repair or regeneration, cosmetic surgery, implants or plastic surgery or other applicable condition or a combination thereof.

In certain embodiments, the present disclosure provides methods for alleviating one or more symptoms and/or for treating inflammatory disease in a subject in need of thereof. In some embodiments, the present disclosure provides a method of treating an inflammatory disease, disorder, or condition by administering to a subject in need thereof a hydrogel described herein having at least one stem cell. In certain embodiments, a hydrogel can further have one or more additional active agents. Such additional active agents can be small molecules or a biologic and can include, for example, acetaminophen, non-steroidal anti-inflammatory drugs (NSAIDS) such as aspirin, ibuprofen, naproxen, etodolac, and celecoxib, colchicine, corticosteroids such as prednisone, prednisolone, methylprednisolone, hydrocortisone, and the like, probenecid, allopurinol, febuxostat, and sulfasalazine. Other examples can include monoclonal antibodies such as tanezumab, anticoagulants such as heparin and warfarin, anticholinergics or antispasmodics such as dicyclomine, beta-2 agonists such as albuterol and levalbuterol, anticholinergic agents such as ipratropium bromide and tiotropium.

In certain embodiments, any of the compositions (e.g., hydrogels) described herein can be used for alleviating and/or treating joint pain or tissue injury. In certain embodiments, the present disclosure provides methods for alleviating one or more symptoms and/or for treating joint pain or tissue injury in a subject in need of the treatment hydrogels disclosed herein, as well as a pharmaceutical composition including such. In other embodiments, hydrogels disclosed herein can be used to restore structural components of a subject such as skin, bone, muscle or other tissue.

In certain embodiments, any of the compositions (e.g., hydrogels) described herein can be used to treat various other diseases, conditions and disorders, including arteriovenous fistulas and malformations including, for example, aneurysms such as neurovascular and aortic aneurysms, pulmonary artery pseudoaneurysms, intracerebral arteriovenous fistula, cavernous sinus dural arteriovenous fistula and arterioportal fistula, chronic venous insufficiency, varicocele, pelvic congestion syndrome, gastrointestinal bleeding, renal bleeding, urinary bleeding, varicose bleeding, uterine hemorrhage, and severe bleeding from the nose (epistaxis), as well as preoperative embolization (to reduce the amount of bleeding during a surgical procedure) and occlusion of saphenous vein side branches in a saphenous bypass graft procedure, among other uses. In some embodiments, hydrogels described herein can be used to treat a cancer. In other embodiments, hydrogels described herein can be used to treat a cancer having a solid tumor. In some embodiments, hydrogels described herein can be used to treat a solid tumor by blocking blood supply to the tumor or delivery by products of cells carried by hydrogels disclosed herein.

In certain embodiments, any of the compositions (e.g., hydrogels) can be injectable compositions wherein the hydrogels herein can be used to deliver one or more therapeutic agents (e.g., active agents) locally to treat any number of diseases, disorders, and conditions treatable by local drug delivery.

In some embodiments, to perform methods disclosed herein, a therapeutically effective amount of the hydrogels or a pharmaceutical composition including such can be administered to a subject who needs treatment via a suitable route (e.g., parenchymal injection, intra-articular injection) at a suitable amount as disclosed herein. In some embodiments, hydrogels or a pharmaceutical composition including such can be administered to a subject by injection through a syringe, a catheter, a trocar, a cannula, and the like.

In some embodiments, to perform the methods disclosed herein, a therapeutically effective amount of the hydrogels or a pharmaceutical composition including such can be administered into at least one site of inflammatory disease. In some embodiments, to perform the methods disclosed herein, a therapeutically effective amount of the hydrogel or a pharmaceutical composition including such can be administered into at least one site of joint pain.

In other embodiments kits are provided for generating, transporting, storing and using hydrogels disclosed herein. In some embodiments, kit for therapeutic use as described herein can include one or more containers further including a composition (e.g., hydrogels) or agents for creating a hydrogel contemplated herein as described herein, formulated in a pharmaceutical composition.

In some embodiments, the kit can additionally include instructions for making, storing or using hydrogels in any of the methods described herein. Instructions can include a description of administration of the hydrogels or a pharmaceutical composition including such to a subject to achieve the intended activity in a subject. The kit can further include a description of selecting a subject suitable for treatment based on identifying whether the subject is in need of the treatment. The instructions relating to the use of the hydrogels as described herein generally include information as to dosage, dosing schedule, and route of administration for the intended treatment.

In some embodiments, kits are provided for generating any of the hydrogels as described herein. In other embodiments, a kit can contain at least two of the polymer backbones described herein. In some embodiments, a kit can include instructions on how to combine the two of the polymer backbones to form a hydrogel, how to increase viscosity of the hydrogel, how to increase stress relaxation of the hydrogel, or a combination thereof.

In certain embodiments, the containers can be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, an infusion device. A kit can have a sterile access port (for example, the container can be an intravenous solution bag or a vial having a stopper pierceable by a hypodermic injection needle). The container can also have a sterile access port. Kits optionally can provide additional components such as buffers and interpretive information. In some embodiments, the kit can have a container and a label or package insert(s) on or associated with the container. In some embodiments, the disclosure provides articles of manufacture including contents of the kits described above.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of the invention.

### Example 1

In one exemplary method, hydrogel compositions were engineered and characterized. In this example, hydrogels cross-linked with covalent adaptable hydrazone bonds (formed between an aldehyde and hydrazide group) were formed and the resulting gel was stabilized with a slow reacting strain which promoted azide alkyne cycloaddition between an 8-arm poly(ethylene glycol) (PEG) functionalized with bicyclononyne and pendant azides on hyaluronic acid (HA).

### Formation of Macromers

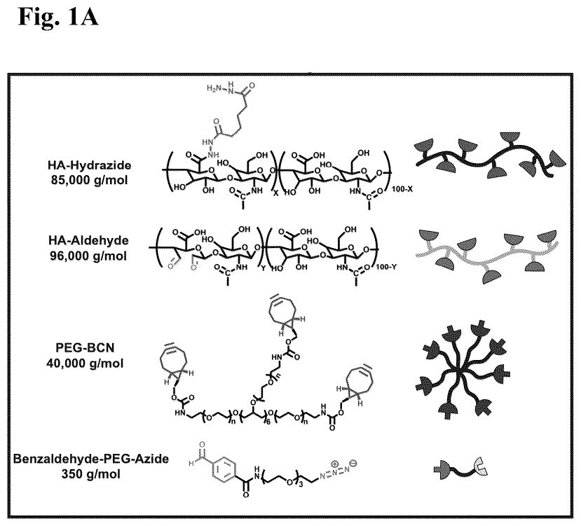

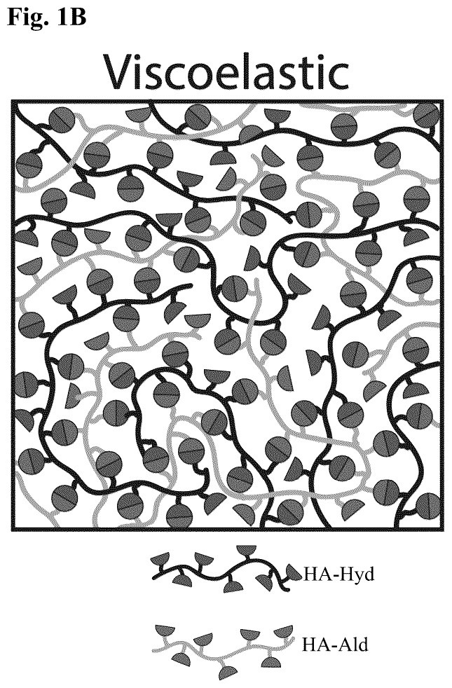

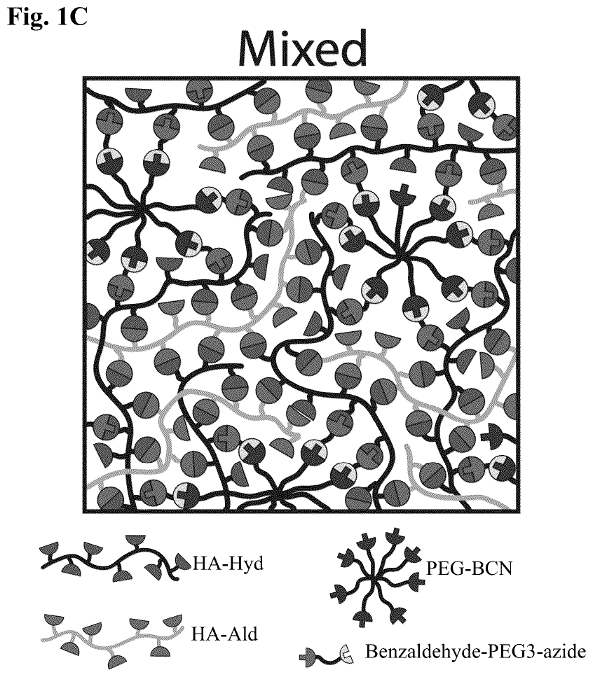

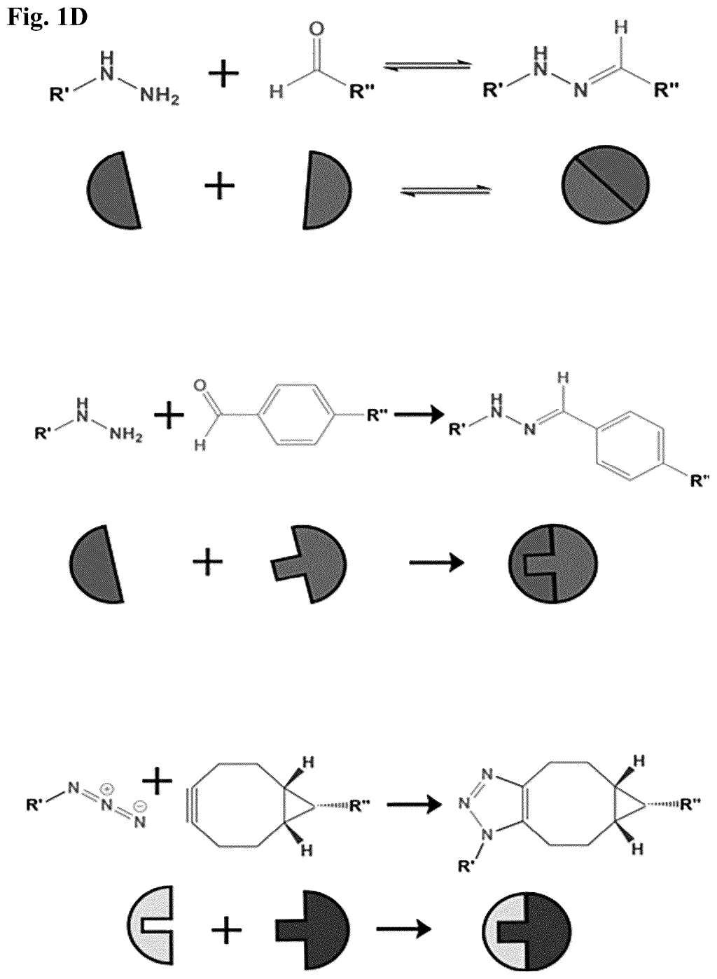

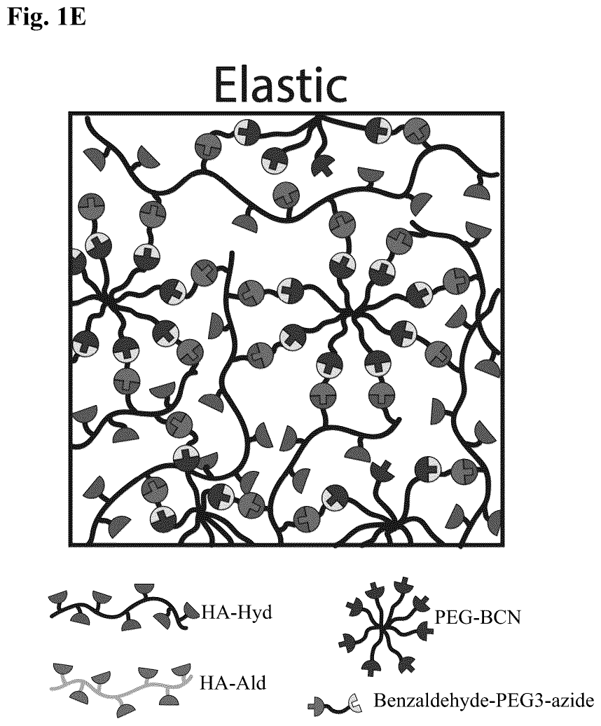

In certain exemplary methods, hyaluronic acid (HA) was functionalized with either an aliphatic aldehyde (HA-Ald) or hydrazide (HA-Hyd). In brief, HA-Ald was synthesized from hyaluronic acid (HA, MW=400 kDa, 74 kDa) where 200 mg of HA and sodium periodate (103 mg, 1:1 periodate/HA molar ratio) were dissolved in 20 mL dH₂O. The reaction was stirred for 2 hours in the dark and subsequently quenched in 2 mL ethylene glycol. The solution was then dialyzed for 5 days against dH₂O (14000 molecular weight cut-off (MWCO)) and lyophilized. HA-Ald was stored under nitrogen at −20° C. until use. Functionalization was determined using a 2,4,6-Trinitrobenzene Sulfonic Acid assay. Briefly, HA-Ald was dissolved at 2 wt % and subsequent reacted with tert-Butyl carbazate (t-Bc, 1% in trichloroacetic acid) in dH₂O overnight. The next day, HA-ALD/t-BC and t-BC standards were reacted with 2,4,6-trinitrobenzenesulfonic acid (TNBS, 6 mM in 0.1 M sodium tetraborate, pH 8) for 1 hour. Samples were then reacted with 0.5 N hydrochloric acid and absorbance was measured at 340 nm on a microplate reader (Tecon). Molecular weight (Mn) was quantified using gel permeation chromatography as follows. 3% (w/w) HA-Ald solution in 10 mL ultrapure water was transported through a gel permeation chromatography (GPC)/size exclusion chromatography (SEC) column with sizes of 500 Å and 250-2000 Å and its molecular weights were measured with a refractive index detector (FIG. 1A).

HA-Hyd was synthesized from HA (MW=400 kDa, 74 kDa) where 1 gram of HA and adipic acid dihydrazide (˜13 g, >60× molar excess) were dissolved in 200 mL dH₂O. 1-Ethyl-3-(3-dimethylaminopropyl)carbodiimide (EDC, 1.55 g, 10 mmol) and hydroxybenzotriazole (HOBt, 1.53 g, 10 mmol) were sere separately dissolved in a DMSO/dH₂O mixture (1:1) and added dropwise to the HA solution. pH was adjusted to 6.8 every 30 minutes for 4 hours, followed by reaction for 24 hours. The solution was dialyzed against dH₂O (3500 MWCO) for 3 days after which products were precipitated in cold acetone and dialyzed again for a week. Products were lyophilized and stored under nitrogen at −20° C. for use. Proton nuclear magnetic resonance spectroscopy (′H NMR, Bruker DMX 360 MHz) was used to characterize the final product. Molecular weight (Mn) was quantified using gel permeation chromatography using the method described above. (FIG. 1A).

Next, an 8-arm poly(ethylene glycol) (PEG, 40 kDa) was functionalized with bicyclononyne ((1R,8S,9s)-bicyclo[6.1.0]non-4-yn-9-ylmethyl (BCN))(PEG-BCN). In brief, 8-arm PEG (˜1 g) and (1R,8S,9s)-bicyclo[6.1.0]non-4-yn-9-ylmethyl (2,5-dioxopyrrolidin-1-yl) carbonate (BCN-OSu; 175 mg) were dissolved in dimethylformamide (5 mL). N,N-Diisopropylethylamine (277 μL, 207 mg, 1.6 mmol, 4×) was added to the mixture, and the reaction was stirred overnight, concentrated, dissolved in water, dialyzed (molecular weight cutoff ˜2 kDa), and lyophilized. Functionalization was confirmed to be >95% by ¹H-NMR by comparing integral values for characteristic BCN peaks (δ 2.24, 1.57, 1.34, 0.92) with those from the PEG backbone (δ 3.63). (FIG. 1A).

Benzaldehyde-PEG3-azide is a crosslinker containing an azide group and a benzaldehyde group. (FIG. 1A).

### Formation of Dual Gel Networks

In other exemplary methods, macromers were dissolved in PBS at stoichiometric ratios. A first gel network system, a HA-hydrazone gel system, was formed by mixing the HA-Ald and HA-Hyd macromers. When these macromers are combined, the hydrazide and aldehyde groups functionalized on the Hyaluronic Acid backbone form hydrazone bonds (FIGS. 1B and 1D).

A second gel network system, PEG-triazole gel system, was formed by mixing the PEG-BCN and benzaldehyde-PEG3-azide macromers. Because BCN is functionalized on an 8-arm PEG it introduced its own backbone. The network formed between the (1R,8S,9s)-Bicyclo[6.1.0]non-4-yn-9-ylmethyl (BCN) and Benzaldehyde-Peg3-azide (Azide) groups, formed an irreversible bond and provided stability. (FIGS. 1C and 1D).

The HA-hydrazone gel system resulted in a viscoelastic gel whereas the PEG-triazole gel system contributes to a more elastic gel. A highly elastic gel is highly stable but has poor injectability whereas a highly viscoelastic gel is easier to inject but has poor stability in solution once injected. Neither of the dual gel networks required an external activator (i.e., temperature, UV light exposure, chemical reaction, photoinitiation, enzymatic reaction, ect.) for bond formation.

### Formation and Characterization of Hydrogels

In certain exemplary methods, for dual gel networks, each polymer, HA-Hydrazide, HA-aldehyde, PEG-BCN, and benzaldehyde-PEG3-azide, were dissolved in PBS. Hydrogels were formed at stoichiometric ratios by first combining HA-hydrazide (ranging from 12% to 100% of the functional arms) and benzaldehyde-PEG3-azide while subsequently combining HA-aldehyde and PEG-BCN, when combined together a hydrogel formed. When combined, the benzaldehyde-PEG3-azide reacted onto the hyaluronic acid backbone with hydrazide, via the hydrazide and benzaldehyde reacting to form an almost irreversible bond (FIGS. 1D and 1E). While the bond can be reversed, the time scale for degradation of this bond was on the order of months.

This reaction effectively converted hydrazide functional groups along the HA backbone into azide moieties for subsequent participation in strain promoted azide-alkyne cycloaddition (SPAAC) reactions with a bicyclononyne (BCN) functionalized 8-arm PEG macromer (FIG. 1D). This SPAAC reaction formed a stable triazole bond that influenced the overall viscoelastic properties of the resulting hydrogel and significantly enhanced the stability of the formulation. By incorporating this more stable crosslink, at the predicted Flory-Stockmayer percolation threshold of the hydrogel, which was calculated to be 1.26% (eqn. 1),

$${p}_{c} = \frac{1}{\sqrt{r ( {f}_{N u} - 1 ) ( {f}_{El} - 1 )}} \qquad ( 1 )$$

based on an 81 arm HA-hydrazide and an 88 arm HA-aldehyde, a stable material was achieved. Changing the proportion of alkyl to benzyl hydrazone bonds allowed the experimenter to tune the material to a range of compositionally-defined viscoelastic responses.

A viscoelastic hydrogel formed when reacting the HA-Aid and HA-Hyd macromers alone; however, without further stabilization, these materials were not stable for longer timescales required for MSC encapsulation and culture (FIG. 1B). Upon addition of the benzaldehyde-PEG-azide crosslinker, the hydrazide functionality was effectively converted into an azide functionality capable of participating in the SPAAC reaction to stabilize the aforementioned viscoelastic hydrogel, which dramatically changed the stress relaxation behavior (FIG. 1C). Therefore, by altering the crosslinker composition, a broad range of viscoelasticity properties were achieved within the hydrogel, and the stress relaxation was characterized. For comparison, a more elastic, stable, hydrogel was synthesized by reacting the HA-Hyd, functionalized with the benzaldehyde-PEG-azide crosslinker, and PEG-BCN to form a SPAAC network (FIG. 1E).

The hydrogel formed from the combination of the dual gel networks did not require an external activator (i.e., temperature, UV light exposure, chemical reaction, photoinitiation, enzymatic reaction, ect.) for bond formation.

The ratio of the viscoelastic hydrazone bonds to elastic triazole bonds was used to yield hydrogels with varying degrees of viscoelasticity and stress relaxation (SR) over time. Specifically, hydrogels with 100%, 88%, 75%, 50%, 25% and 0% of crosslinks comprised of fast-relaxing alkyl hydrazone bonds were fabricated. The gelation time, final storage modulus, viscoelasticity, and stress relaxation time of the hydrogels were characterized in-situ using a parallel plate rheometer (DHR-3). The shear storage modulus of the dual gel networks (HA-Hydrazone:PEG-triazole) measured after equilibration was ˜400 Pa across all conditions.

Hydrazone bonds formed rapidly at physiological pH and created a scaffold that contained the macromers to stabilize the network over time and enable injection. The hydrazone bonds formed at physiological pH faster than could be captured when loading the sample, as observed by the immediate gelation of the hydrogel (G′>G″) for the initial viscoelastic condition (HA-Ald and HA-Hyd only).

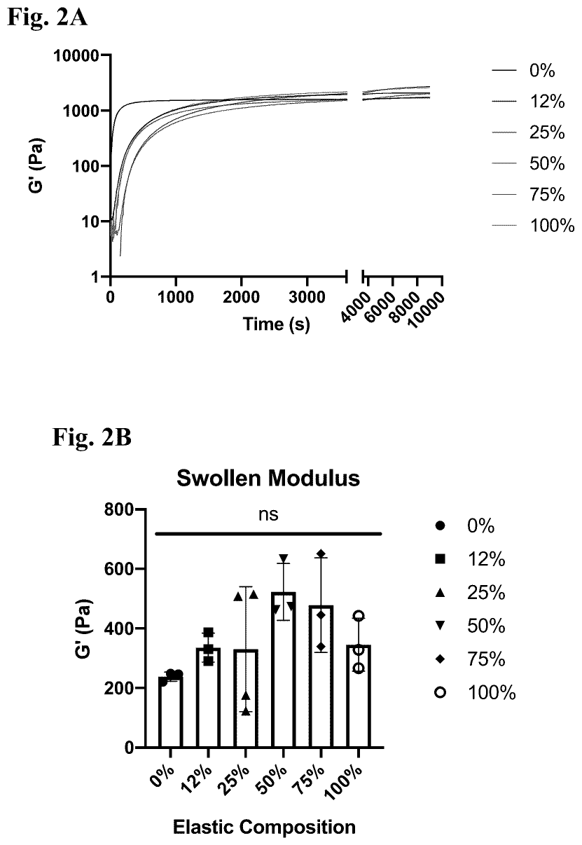

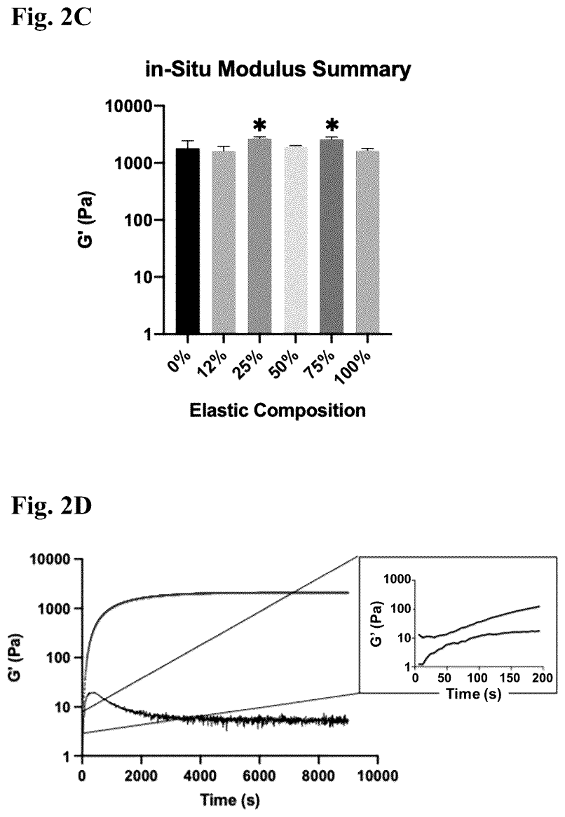

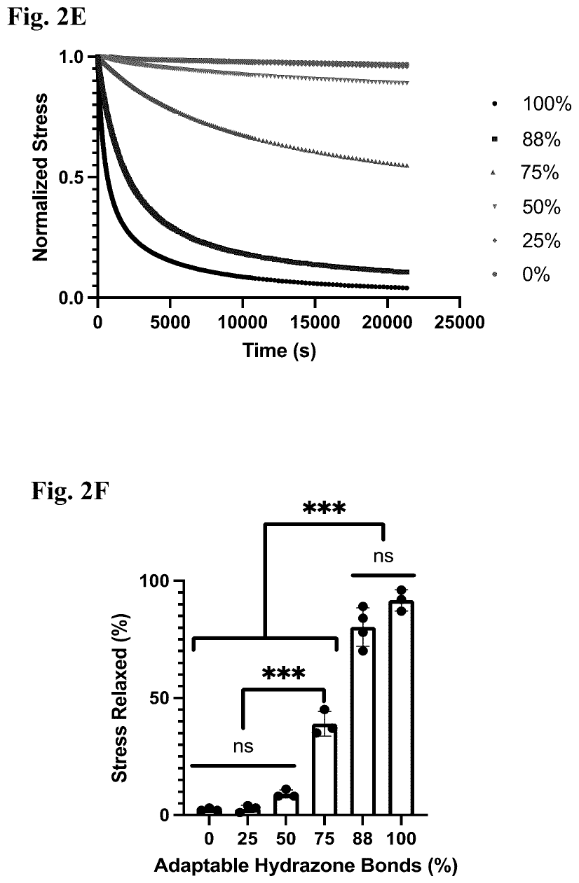

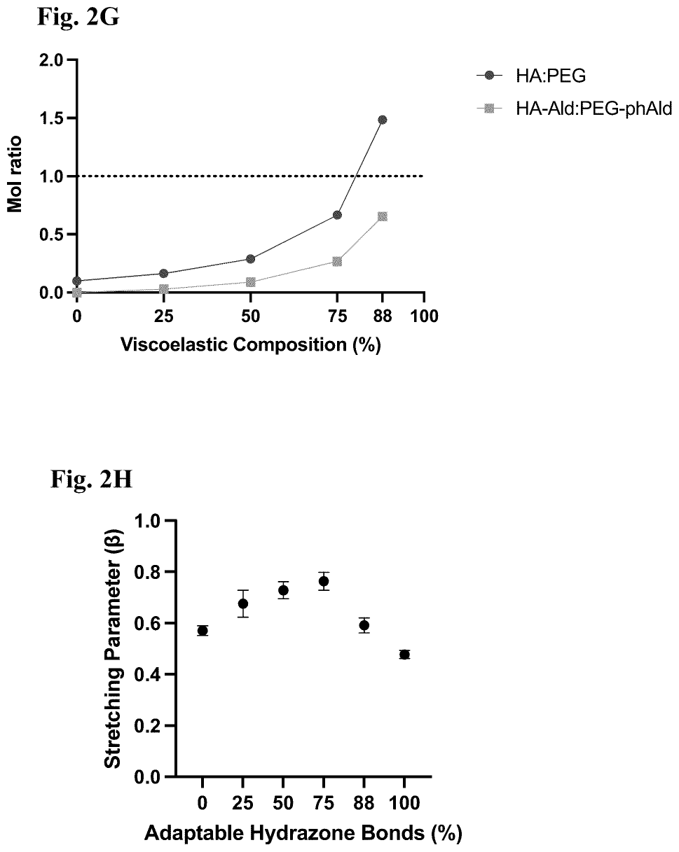

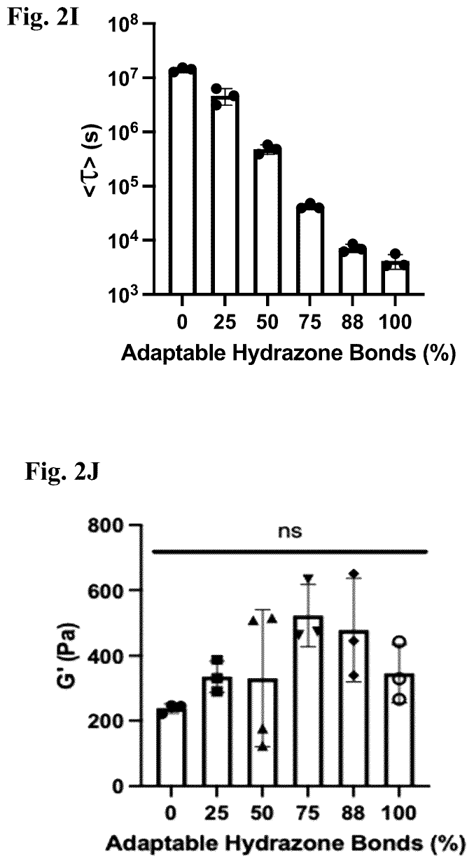

With the addition of the slower-gelling SPAAC crosslinks, the crossover point of G′ and G″ still occurred relatively rapidly where the elastic gel (combination of the dual gel networks HA-Hydrazone and increasing concentrations of PEG-BCN) began to form after about minutes whereas the viscoelastic gel (HA-hyd and HA-ald) forms almost instantaneously (FIG. 2A), reaching steady state network formation after ˜9000 seconds (2.5 hours). The final shear storage modulus of the various hydrogel formulations ranged from 1600 Pa to 2400 Pa (FIG. 2A).

After 24 hours, there was no significant difference in modulus observed between the differing gel conditions (FIG. 2B). All gels had an in situ modulus of less than about 10,000 Pa where gels having viscoelastic compositions of 25% and 75% had a significantly higher in situ modulus compared to the other gels examined (FIG. 2C). Because the hydrazone bonds in the HA-Hydrazone gel system form rapidly, a scaffold is created that allows for the components of the HA-hydrazone-azide and PEG-BCN gel system to stabilize the network over time and enable injection.

Following complete network formation, the stress relaxation properties of the hydrogels were tested. A 10% strain was applied over 1 second and held constant for a period of 6 hours while the relaxation of the applied shear stress was monitored for each hydrogel condition (FIG. 2D). The amount of stress relaxation varied with the percent of alkyl hydrazone bonds present within the hydrogel, ranging from ˜90% to only 2% of stress relaxation for the 100% vs. 0% alkyl hydrazone formulations, respectively (FIGS. 2D-2E). As the functionalization of the HA-Hyd with the azide crosslinker was increased from 0-100%, fewer alkyl hydrazone crosslinks were present, changing the viscoelastic properties of the hydrogel. Further analysis of the stress relaxation data indicated that the 100% and 88% alkyl hydrazone bonds conditions show no significant difference in the percent of stress relaxation, with both relaxing at >80% of the applied stress (FIG. 2F). As the alkyl hydrazone crosslinking content further decreased, major differences in the stress relaxation properties were observed. Specifically, every sample relaxed <50% of the applied stress, with the 50%, 25% and 0% formulations relaxing 10% or less of the applied stress (FIG. 2E). These results suggested a sharp decline in the stress relaxing capabilities of the hydrogel, as fewer dynamic bonds are present in the network and the molar ratio of PEG to HA significantly exceeds 1 (FIG. 2G).

All stress relaxation curves were normalized to their initial values and the data were fit to a Kohlrausch-Williams-Watts function (eqn. 2):

$$\frac{σ}{{σ}_{0}} = {e}^{- {( \frac{t}{{τ}_{k}} )}^{β}} \qquad ( 2 )$$

In this equation, the normalized stress, σ/σ₀, was modeled as an exponential decay with the time constant, τₖ, that exists in a distribution described by the stretching parameter, β. Hydrogel stress relaxation encompasses a broad range of relaxation time constants due to compositional and topological heterogeneities (i.e., multiple covalently adaptable chemistries, polymer chain entanglements and loops, and differing polymer network backbones) that exist within a hydrogel network. The Kohlrausch-Williams-Watts function was used as an empirical relationship to describe the relaxation behavior of samples who exhibit broadly heterogenous relaxation timescales. The heterogeneity of the relaxation time constants were qualitatively understood through β, as a value of 1 representing a singular relaxation time constant and deviation towards 0 representing a broadening of this distribution (0<β<1). The model parameters are summarized in FIG. 2H which illustrates the stretching parameters for each hydrogel composition of the exemplary method. The stretching parameters had a moderate degree of heterogeneity with β>0.5 for most conditions.

Once the time constant and stretching parameter were known, an average relaxation time constant, ( [symbol] τ [symbol] ), was calculated by integrating the model over the entire time domain (t=0 to t=∞) (eqn. 3):

$$〈 τ 〉 = \frac{X τ}{β} Γ ( \frac{1}{β} ) \qquad ( 3 )$$

A characteristic relaxation time for each hydrogel composition was calculated using equation 3 and parameters fit to the stress relaxation data. These characteristic relaxation times were found to span four orders of magnitude ( [symbol] τ_{88%} [symbol] =4.2×10³ s to [symbol] τ_{0%} [symbol] =1.4×10⁷ s) representing precise control over the timescale of relaxation within the hydrogels that can range from roughly one and a half hours to 6 months (FIG. 2I).

In certain exemplary methods, to determine the equilibrium swollen shear storage modulus of each hydrogel formulation, the samples were swollen in PBS for 24 hours and subsequently analyzed with rheology according to methods detailed herein. The final equilibrium storage modulus (G′) was found to be ˜400±150 Pa across all conditions, with no significant difference between any of the conditions (FIG. 2J). Therefore, for cell culture experiments using these formulations, the primary difference between hydrogel properties was stress relaxation behavior.

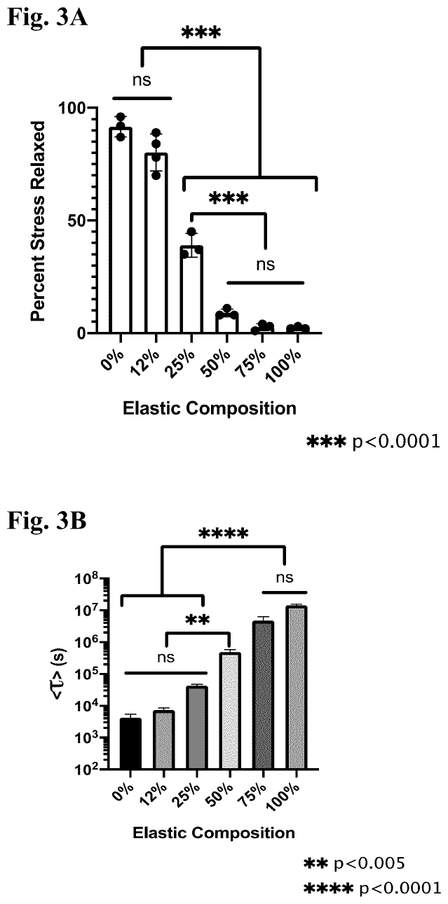

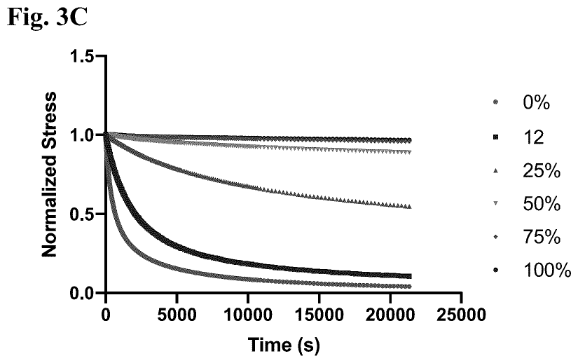

Using shear rheology, one test used to measure the viscoelastic properties of hydrogels was the stress relaxation test. In this test, a constant strain was applied, and the responding stress was measured over time. An elastic material would maintain a constant stress, while viscoelastic materials exhibit stress relaxation. The percent of stress relaxation over time was measured and ranged from 92% to 2%, depending on the PEG-triazole content from 100% to 0%, respectively (FIGS. 3A and 3B). Specifically, 0% PEG-triazole content relaxed 92% of the stress, 12% PEG-triazole content relaxed 84% of the stress, 25% PEG-triazole content relaxed 39% of the stress, 50% PEG-triazole content relaxed 9% of the stress, 75% PEG-triazole content relaxed 3% of the stress, and 100% PEG-triazole content relaxed 2% of the stress. Stress of the gels was normalized over time (FIG. 3C).

These data illustrate that with an increasing amount of PEG-triazole dosed into the gel (increasing elasticity) the stress relaxation of the gel decreased significantly, whereas the time constant for stress relaxation increased as elasticity of the gel increased. All gel formulations demonstrated similar swollen shear storage modulus values after 24 hours. This illustrated that the ratio of HA-Hydrazone to PEG-triazole can be tuned to control viscoelasticity and stress relaxation within the gel over time while the bulk shear storage modulus of the gel remains unchanged.

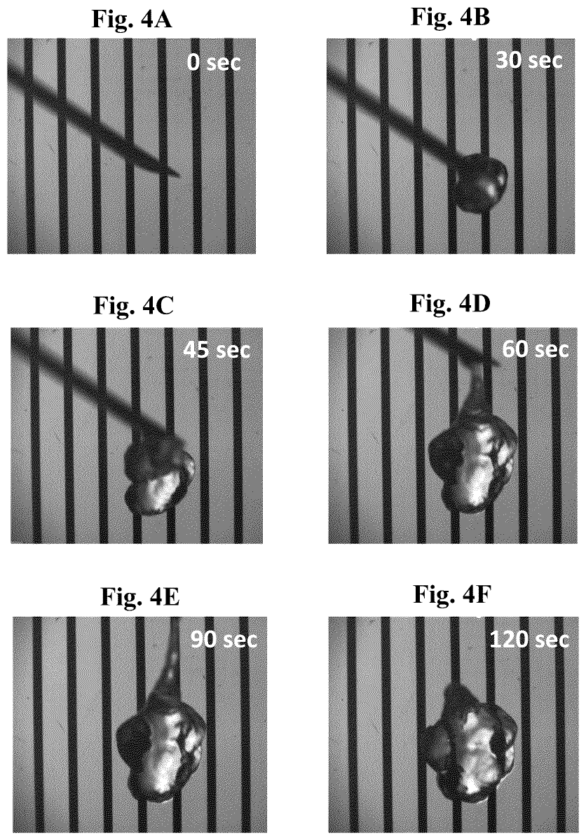

In another exemplary method, to determine if the unique dual gel network disclosed in this example can allow for highly elastic gels to be injectable, time lapse vides were taken of gels having increasing concentrations of PEG-triazole content (and thus increasing electability) being ejected from a standard injection syringe. The 100% PEG-triazole elastic gel control could not be ejected from the injection syringe. However, gels having 75% PEG-triazole and 25% HA-hydrazone were able to be ejected from the syringe and maintain a gel form after ejection (FIGS. 4A-4F).

### Example 2

In another exemplary method, mesenchymal stem cells (MSCs) were encapsulated in gels with different adaptability, and the MSC secretome profile was measured as a function of the material properties. First, rat MSCs were cultured in low-glucose Dulbecco's Modified Eagle supplemented with penicillin streptomycin, fungizone and 10% FBS. MSCs between passages 5-6 were used in the exemplary method disclosed. Next, hydrogels having a combination of the gel networks HA-hydrazone and PEG-triazole were prepared as described in Example 1 and were referred to in this example as “PEG-HA dual networks.” MSCs were encapsulated in the PEG-HA dual networks at a density between 1-5 million cells/mL. MSC morphology was quantified by immunostaining the nuclei (DAPI) and actin cytoskeleton (Rhodamine Phalloidin) and imaged using a laser scanning confocal microscope.

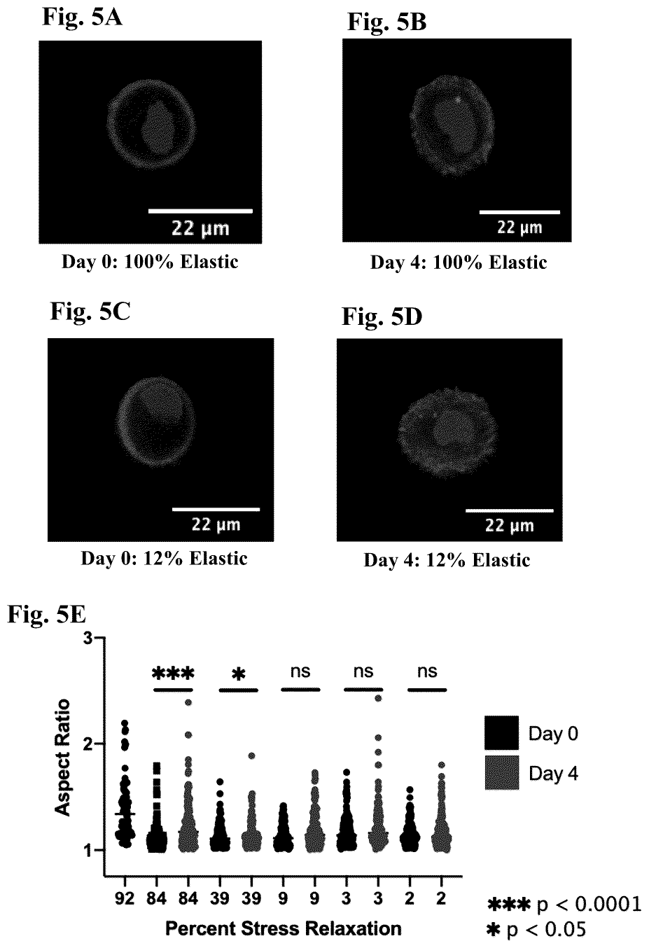

After 0 and 4 days in culture, the morphology of MSCs encapsulated in the PEG-HA dual networks was quantified by immunostaining the nuclei (DAPI) and actin cytoskeleton (Rhodamine Phalloidin) and imaged using a laser scanning confocal microscope. FIGS. 5A and 5B show morphology of MSCs encapsulated in a PEG-HA dual network gel having 0% viscoelastic composition (100% PEG-triazole and 0% HA-hydrazone) after 0 days (FIG. 5A) and 4 days (FIG. 5B). FIGS. 5C and 5D show morphology of MSCs encapsulated in a PEG-HA dual network gel having 88% viscoelastic composition (12% PEG-triazole and 88% HA-hydrazone) after 0 days (FIG. 5C) and 4 days (FIG. 5D). FIG. 5E illustrates MSC morphology characterized as a function of stress relaxation. The 92% SR condition had completely degraded by day 4, while the 84% and 39% SR conditions both demonstrated significant cell spreading, with embodiment ratios of 1.25 and 1.20, respectively. MSCs in the 9%-2% SR formulations remained rounded, with embodiment ratios less than 1.20.

### Example 3

In another exemplary method, rat mesenchymal stem cells (rMSCs) were encapsulated at a density of 1 million cells/ml into the same HA-PEG hydrogel formulations now containing 1 mM of the RDG peptide ligand, KRGDS (SEQ ID NO: 8). The RGD was attached to the HA-Hyd as it was functionalized with a benzaldehyde group and therefore able to interact with the rMSCs. First, rMSC viability was measured using Calcein AM and ethidium homodimer live/dead stains. After 24 hours, >85% viability was measured across all samples (FIG. 6A). Next, rMSC morphology was investigated as a function of matrix stress relaxation (FIG. 6B). After 4 days of culture, rMSC-laden hydrogels were fixed and stained to visualize the cytoskeletal morphology (F-actin, green) and nuclear shape (Dapi, blue) with a confocal microscope (FIG. 611). Over the course of the experiment, the 100% viscoelastic formulation was completely degraded, presumably by a combination of rMSC-secreted proteases and potential reaction of the aldehydes with serum proteins in the medium, both of which effectively break crosslinks. This observation further emphasized the need to stabilize the purely hydrazone materials for longer term cell studies. In the 100% SPAAC (elastic) hydrogel, the rMSCs remained relatively rounded, with the embodiment ratio remaining close to 1.0, with no significant difference from day 0 to day 4 (FIG. 6C). In contrast, rMSCs in the highest viscoelastic condition, 88%, were significantly larger, more spread, and elongated by day 4. Over the time course of culture from day 0 to day 4, the cellular embodiment ratio increased to greater than 1.20 due to the physical remodeling of the surrounding networks in the 88% viscoelastic hydrogel. Interestingly, rMSCs in the 25, 50, and 75% viscoelastic conditions remained largely rounded, and did not show significant spreading by day 4 (FIGS. 6C-6G). This led to a critical amount of viscoelasticity, or amount of pericellular crosslinks needed to rearrange, in order to influence morphology. This was likely due to rMSCs responding to networks with faster stress relaxation on the order of two hours compared to longer relaxation times as the other conditions demonstrated embodiment ratios of less than 1.20 after 4 days (FIG. 6H).

In these exemplary methods, across all conditions, using rMSCs encapsulated in about 88% alkyl hydrazone crosslinks (e.g., highly adaptable, but stable for longer-term culture), demonstrated significant spreading by Day 4, as compared to certain other conditions (FIG. 6J). Over the time course of culture from day 0 to day 4, the cellular aspect ratio increased from roughly 1.0 to greater than 1.60±0.18 and was indicative of physical remodeling of the surrounding network. rMSCs encapsulated in the 0, 25, 50, and 75% alkyl hydrazone containing hydrogels remained largely rounded, with aspect ratios of 1.20±0.038 or less, and did not show significant spreading by day 4 (FIGS. 6C-6G and 6J). In contrast, rMSCs in the 88% alkyl hydrazone small clusters were observed; this phenotype was not seen in any other condition (FIG. 6H).

Mechanical sensing as a function of the network adaptability was measured via analysis of the nuclear to cytoplasmic YAP ratio. By day 4, the 88% viscoelastic condition demonstrated greater nuclear localization of YAP, with an average nuclear to cytoplasmic ratio of 6.5±1.3, as compared to the slower stress relaxing conditions of 75% and 0%, which had average nuclear to cytoplasmic ratios of 2.8±1.2 and 2.8±1.3, respectively (FIG. 6K-6L). Along with this, small clusters of cells were again observed by day 4 in the 88% viscoelastic condition and stained for YAP (FIG. 6M). Additionally, cellular, and nuclear volumes were analyzed at day 4 for the 88%, 75% and 0% viscoelastic conditions to better understand the effects of YAP localization. There was a striking increase in cell volume in the 88% alkyl hydrazone condition as compared to the 75% and 0% alkyl hydrazone conditions, with an average cell volume of ˜6100±4500 μm³, as compared to ˜3200±1700 μm³ and 3500±1600 μm³, respectively (FIG. 6N). However, nuclear volumes remained consistent across all conditions, with an average volume of ˜1200 μm³ (FIG. 6O).

With the drastic increase in cellular volume, the YAP nuclear to cytoplasmic ratios were better understood. As YAP signal becomes more nuclear while the nuclear volume remained relatively the same across all conditions, intensity in the nucleus of YAP increased; in contrast, intensity in the cytoplasm decreased as the cytoplasmic volume increases (with increasing alkyl hydrazone bonds and HA content). The change in cellular morphology and YAP in the 88% alkyl hydrazone condition suggested a critical proportion of adaptable crosslinks in the pericellular region to allow for viscoelasticity-induced cell spreading and morphological changes. Specifically, rMSCs responded to networks that had fast stress relaxation timescales (˜2 hours), which allowed for microenvironmental rearrangements and lead to changes in morphology and clustering. The 88% alkyl hydrazone bond condition demonstrated significant cell spreading and YAP nuclear localization. This was explained by the molar ratios of HA to PEG, recalling that the 88% adaptable hydrazone bond condition had a HA to PEG ratio greater than 1 whereas all ensuing conditions had PEG to HA ratios greater than 1. (YAP/TAZ are primary sensors of the cell's physical nature, as defined by cell structure, shape and polarity. YAP/TAZ activation also reflects the cell “social” behavior, including cell adhesion and the mechanical signals that the cell receives from tissue scaffolding and surrounding ECM.)

### Example 4

In another exemplary method, rat mesenchymal stem cell (rMSC) spreading and morphology were assessed. Cell spreading and morphology in covalent adaptable networks requires coordinated relaxation of multiple bonds over the size scale of microns. The rMSCs were metabolically active and can deposit large concentration of matrix molecules, even after short culture periods. As such, matrix deposition was characterized herein as a function of the hydrogels stress relaxation properties. This was conducted via measuring the nascent protein deposition of the rMSCs in rMSC-laden gels. In brief, rMSCs were encapsulated in the HA-PEG hydrogel formulations containing RGD at a cell density of 3 million cell s/ml. Upon encapsulation a noncanonical amino acid L-homopropylargylgylcine (HPG) was added into the media on day 0 and day 2 at a concentration of 100 μM. After 4 days the hydrogels were fixed and immunostained. The HPG was then incorporated into newly synthesized proteins and enabled the visualization of the deposited proteins via a copper catalyzed click-reaction of an azide functionalized fluorophore onto the HPG and the cell membrane was visualized using an HCS cell mask blue stain on a confocal microscope (FIGS. 7A and 7B). rMSCs were encapsulated in the elastic control formulation and the 88% viscoelastic condition. The 88% viscoelastic condition demonstrated extensive protein deposition throughout the pericellular region, especially in comparison to the elastic control, which was much sparser (FIG. 7C). The data was quantified via confocal microscopy and analyzed using the ‘Bond’ plugin in ImageJ. The 88% viscoelastic condition had a mean protein thickness of 1.5±0.34 μm, and an average maximum protein thickness of 2.5±0.62 μm. In contrast, the 100% SPAAC hydrogel had a mean protein thickness of 1±0.36 μm and an average maximum protein thickness of about 1.7±0.92 μm (FIG. 7D). FIG. 7E illustrates the maximum secreted protein thickness for the two extreme conditions of roughly 2.5 microns, and 1.8 microns. Taken together this data demonstrated the ability of rMSC's to build a significant pericellular matrix that encompasses the entire cell in fast stress relaxing hydrogels.

In one method, rMSCs were encapsulated in the elastic control formulation and the 88% viscoelastic condition under the same condition as above except that the hydrogels were fixed and immunostained after 7 days of culture. FIGS. 8A-8C illustrate encapsulated rMSCs formed multinucleated structures in high viscoelastic hydrogels.

Next, matrix deposition was characterized as a function of the hydrogel stress relaxation properties by measuring nascent protein deposition in rMSC-laden gels. Nascent protein was visualized in the hydrogels via the incorporation of a noncanonical amino acid L-homopropylargylgylcine (HPG). rMSCs were encapsulated in all hydrogel conditions: 0%, 25%, 50%, 75%, and 88% adaptable hydrazone bonds per hydrogel. FIG. 9A illustrates a representative image of the visualization of the secreted proteins in the significantly different populations for protein deposition, 88%, 75% and 0% viscoelastic and includes both single cell and clustered nascent protein deposition from the 88% viscoelastic condition.

The 88% alkyl hydrazone condition demonstrated extensive protein deposition throughout the pericellular region, especially in comparison to the elastic control. The 88% alkyl hydrazone condition had a mean protein thickness of 1.45±0.38 μm, and an average maximum protein thickness of 2.31±0.77 μm (FIGS. 9B-9C). The 75% alkyl hydrazone condition had a mean protein thickness of 1.21±0.30 μm and an average maximum protein thickness of about 1.88±0.59 μm, with the 50, 25, and 100% SPAAC hydrogels having a mean protein thickness of 1.05±0.25 μm or less, and an average maximum protein thickness of about 1.7±0.57 μm or less (FIGS. 9B-9C). The total area of deposited nascent proteins indicated that the fastest-relaxing hydrogel (88% alkyl hydrazone) had greatest amount of total nascent protein deposition (FIG. 9D).

The rMSCs in the 88% alkyl hydrazone were separated into two distinct phenotypes: those which clustered together and those that remained as single cells. Cells within these two groups were analyzed for the total amount of nascent protein deposition and rMSCs residing in clusters secreted greater concentration of nascent proteins as compared to single cells (FIG. 9E). To further this analysis, the composition of the fibronectin and collagen content within the nascent proteins deposited was quantified. While minimal collagen was deposited by the encapsulated cells (data not shown), fibronectin was highly secreted and shown to localize in the branches extending from the rMSCs in the 88% alkyl hydrazone condition. In the 0% alkyl hydrazone condition, fibronectin deposition was seen uniformly around the cell (FIGS. 9F-9G). Taken together, this data suggested that rMSCs build a significant pericellular matrix, composed of large concentration of fibronectin, around the cellular niche in fast-relaxing hydrogels.

These data of disclosed herein provide a greater understanding of the impact of the compositionally defined stress relaxation on the formation and composition of the pericellular matrix, which may further contribute towards the cell's ability to mechanosense, as seen through Exo-1 inhibition studies. These studies demonstrated with the inhibition of Exo-1, nascent protein deposition was reduced in both the 88% and 75% adaptable bond conditions, 0.91±0.11 μm and 0.91±0.14 μm, respectively, down to the control level of the 0% adaptable bond conditions of 0.94±0.16 μm (FIGS. 10A-10B). Further analysis of the aspect ratios of the Exo-1 inhibited cells demonstrated a reduction in the rMSC spread morphology, with both the 88% and 75% adaptable bond conditions having aspect ratios now of, 1.27±0.081 μm and 1.22±0.072 μm, respectively, which is similar to that of the 0% adaptable bond control of 1.26±0.034 μm (FIG. 10C). This indicated one possible role for the deposited nascent proteins, as they are necessary for mechanosensing leading to cellular spreading.

### Example 5

In another exemplary method, to assess MSC secretome profiles as a function of the material properties, the secretory profiles of MSCs at day 4 after encapsulation in PEG-HA dual network gels were measured using a Rat Cytokine Array C2 kit. All data was normalized to DNA concentration, with the control media used as the reference array and cytokine secretion of pro- and anti-inflammatory cytokines was characterized as a function of hydrogel stress relaxation. FIG. 11A illustrates a heat map of all the cytokines tested for in the Rat Cytokine Array C2 kit and FIG. 11B illustrates a closer look at the pro- and anti-inflammatory cytokine secretion detected by the array assay.

These data demonstrate that the MSC secretory profile increased in hydrogels that had higher levels of stress relaxation, indicating that viscoelasticity regulated cytokine secretion after 4 day. At day 4, MSCs in the 39% SR condition had the highest levels of cytokine secretion, while MSCs in the 2% SR condition secreted the least. Notably, the anti-inflammatory cytokine interleukin 10 (IL-10) decreased as the stress relaxation of the gel decreased, while the pro-inflammatory cytokine, tumor necrosis factor alpha (TNFa), increased with decreasing stress relaxation. The PEG-HA dual network gel with the 25% Elastic (75% viscoelastic) condition demonstrated the greatest secretion of cytokines. Hallmark cytokine for anti-inflammatory environments, IL-10, was secreted the most from the 25% elastic condition, and the hallmark cytokine for pro-inflammatory environments, TNF-alpha, was secreted the most from the 75% elastic condition. IL-10 and IL-6 were upregulated in a more viscoelastic network and decreased in a stiffer network. Pro-inflammatory cytokine TNF-alpha was more upregulated in a stiffer network.

As disclosed herein, these data demonstrate that novel PEG-HA dual network hydrogels can be tuned to promote MSC cell delivery, spreading and/or MSC cytokine secretion by dosing. For example, dosing to effect stress relaxation (PEG-triazole) of the hydrogel. Promoting MSC cell spreading can increase recovery efforts following injection into a damaged tissue area. The PEG-HA dual network gel contribution to the inflammatory environment at the site of injection can also increase tissue recovery as well as provide localized anti-inflammatory relief to the injection site.

### Methods Used in Examples 1-5

The following methodologies were performed in the exemplary methods disclosed in Examples 1-5 as provided herein.

Synthesis of HA-Aldehyde and Characterization. Briefly, Ha (500 kDa, 500 mg) was dissolved in 50 mL dH₂O, then, sodium periodate (267.5 mg, 1:1 periodate/HA molar ratio) was added into the reaction mixture. The reaction was stirred in the dark for 2 hours and successively quenched in 70 μL ethylene glycol. The reaction was then dialyzed for 3 days against dH₂O (8000 Molecular weight cut-off (MWCO)) and lyophilized. HA-Ald was flash frozen in liquid nitrogen and stored at −20° C. until further use. The functionalization of the HA-Ald macromer was quantified using a 2,4,6-Trinitrobenzene Sulfonic Acid (TNBS) assay. Briefly, the HA-Ald was dissolved at 2 wt % and then reacted with tert-Butyl carbazte (t-BC, in 1% trichloroacetic acid) in dH₂O. After 24 hours, the HA-Ald/t-BC and t-BC standards were reacted with 0.5 ml TNBS (6 mM in 0.1 sodium tetraborate at pH 8) for 1 hour. Samples were then reacted with 0.5 N hydrochloric acid and measured at an absorbance of 340 nm on a microplate reader (˜35% functionalization).

Synthesis of HA-Hydrazide and Characterization. Briefly, Ha (60 kDa, 500 mg) was dissolved in 100 ml dH₂O before adipic acid dihydrazide (˜6.5 mg, >60× molar excess) was added in large molar excess to the reaction and the pH was adjusted to 6.8. 1-Ethyl-3-(3-dimethylaminopropyl)carbodiimide (EDC, 776 mg, 4 mmol) and hydroxybenzotriazole (HOBt, 765 mg, ˜6 mmol) were separately dissolved in DMSO/dH₂O mixture (1:1) and added dropwise to the reaction. The pH was adjusted to 6.8 every 30 minutes for 4 hours and then allowed to react for 24 hours. The solution was dialyzed against dH₂O (8000 MWCO) for 3 days and then lyophilized. Once dried, the products were weighed, dissolved in a 5 wt % NaCl/dH₂O solution, and precipitated into pure ethanol and dialyzed for another 3 days. Products were then lyophilized and flash frozen in liquid nitrogen and stored at −20° C. until use. HA-Hyd was characterized using ¹H NMR (˜40% functionalization).

Synthesis of PEG-BCN and Characterization. Briefly, an 8-arm 40 kDa PEG-amine (1.0 g, 0.2 mmol amine) and BCN-oSu (0.1 g, 0.343 mmol, 1.7×) were added to a 50 ml round bottom (RB) flask and dried overnight at 80° C. A minimal amount of dry DMF was added to the RB flask to dissolve the contents (10 ml). The flask was then placed under an argon gas atmosphere and stirred at room temperature. N,N-Diisopropylethyleamine (0.8 mmol, 4×) was added to the solution and the reaction was left to proceed overnight. The following day, the reaction mixture was diluted with dH₂O and dialyzed for 3 days (8000 MWCO). The dialyzed solution was lyophilized for 3 days, resulting in a dried white powder as the PEG-BCN product (0.985 g, 98% yield). End group functionalization was confirmed using ¹H NMR (>95% functionalization).

Peptide Synthesis. Benzaldehyde-KGRGDS (SEQ ID NO: 7) was synthesized using standard Fmoc chemistry and Rink Amide MBHA resin on a Protein Technologies Tribute Peptide Synthesizer. Briefly, a peptide cleavage solution was formed by dissolving dithiothreitol (DTT) and phenol (1:1) in a solution of 95% trifluoroacetic acid (TFA), 2.5% triisopropylsilane (TIPS), and 2.5% deionized water. The synthesized peptides were cleaved for 2 hours. Cleaved peptides were precipitated in cold diethyl ether, recovered by centrifugation, and desiccated overnight. The cleaved peptides were then purified by reverse-phase HPLC (Waters Delta Prep 4000) purification on a C18 column using a linear acetonitrile:water gradient. The collected fractions of purified peptides were identified by matrix-assisted laser desorption/ionization-time-of-flight (MALDI-TOF) mass spectrometry.

Hydrogel Formation and Rheological Characterization. Functionalized HAs were dissolved in phosphate buffered saline (PBS, pH 7.4) at 3 wt %. Functionalized PEG-BCN was dissolved in PBS at 10 wt % and the azide-PEG3-phenolaldehyde (BroadPharm) crosslinker was dissolved at a concentration of 20 mM. Hydrogels were formed with 3 w/v % final polymer content based on stoichiometry. In situ rheology measurements were performed using a TA Instruments DHR-3 rheometer with an 8 mm parallel plate geometry. Hydrogel formation was evaluated by time sweeps (1.0 Hz; 0.5% strain). For stress relaxation experiments, 6 hours of measurement followed 10% strain applied over 1 second, as dictated by the amount of time for the 100% viscoelastic condition to relax the majority of the applied stress (>90%). Formulations were defined by varying the percentage of alkyl hydrazone bonds present within the hydrogel by modifying the HA-hydrazide with a small molecule, benzaldehyde-PEG-azide, which effectively converts the HA-hydrazide to an azide. The formulations consisted of 100% (purely alkyl hydrazone), 88% alkyl hydrazone (i.e., 12% of the functional HA-hydrazide arms were functionalized with benzaldehyde-PEG-azide), 75%, 50%, 25%, and 0% (purely SPAAC, i.e., 100% of the functional HA-hydrazide arms were functionalized with benzaldehyde-PEG-azide), incorporating the necessary proportions of PEG-BCN and HA to achieve these functionalities. Mineral oil was applied to the hydrogel perimeter to prevent evaporation during the experiment. Theoretical models were fit using the curve fit application in MATLAB.

Cell Culture and Encapsulation. Sprague-Dawley (SD) rat mesenchymal stem cells (rMSCs) were expanded and cultured. Briefly, the cells were obtained at passage 2 and expanded to passage 5 in growth medium specifically, Dulbecco's Modified Eagle Medium of low glucose (1 ng/ml glucose) supplemented with 10% FBS, 50 μg/ml streptomycin, and 0.5 μg/ml of Amphotericin B. The media was changed after 24 hours, and then every three days until 80-90% cell confluency was reached. When the desired cell confluency was reached, the cells were stored at passage 5 in liquid nitrogen. Hydrogels were fabricated by pre-reacting the HA-Hyd with the benzaldehyde-PEG-azide crosslinker, and KGRGDS (SEQ ID NO: 7) based on stoichiometry for the desired 3 wt % hydrogel formulation, of 100% (purely alkyl hydrazone), 88%, 75%, 50%, 25%, and 0% (purely SPAAC), and allowing them to react overnight. The following day, the HA-Ald, PEG-BCN, and PBS were mixed at stoichiometric ratios in a modified syringe barrel. rMSCs at passage 5 were thawed and placed in growth medium. The rMSC suspension was centrifuged (200 rcf, 5 minutes) and the resulting pellet was resuspended in the HA-Hyd, benzaldehyde-PEG-azide, KRGDS mixture for a cell density of 1-5 million cells/ml. The cell suspension was then mixed into the syringe barrel with the HA-Ald, PEG-BCN and PBS. The hydrogels were allowed to react until a soft gel formed (5 minutes or less) and then placed onto either benzaldehyde functionalized coverslips (for the 100, 88, and 75% conditions) or azide functionalized coverslips (for the 50, 25 and 0% conditions), and allowed to react for an additional 2 minutes before 1 ml growth medium was added.

Cell Viability, Morphology. and YAP. For viability assays, cells were stained with Calcein AM/ethidium homodimer Live/Dead solution. To analyze morphological changes and YAP (yes-associated protein 1), rMSCs grown in the HA-PEG hydrogels were fixed with formalin (30 min, room temperature) following 4 days of growth and then rinsed with PBS. After fixation, the hydrogels were permeabilized with 0.1% Triton X-100 in PBS (1 hour, room temperature) and blocked using 5% BSA (in PBS (1 hour, room temperature). The samples were incubated with DAPI (1:1000) and Alexa Flour 647 Phalloidin (1:300) in blocking buffer (overnight, 4° C.) (morphology) or incubated with DAPI (1:1000), Alexa Flour 647 Phalloidin (Invitrogen, 1:300) and YAP (1:500) in blocking buffer (YAP nuclear:cytoplasm ratio). After washing with PBS with 0.05% Tween 20 (PBST) three times to remove excess stain, the fluorescently labeled rMSCs were imaged using confocal microscopy (Zeiss LSM 710). ImageJ (NIH) was used to visualize and quantify morphological changes using the analyze particles plugin. Imaris was used to visualize and quantify YAP changes.

Nascent Protein Deposition. Cells were encapsulated at a concentration of 3 million cells/ml and cultured in glutamine-, methionine- and cystine-free high glucose DMEM with mM cystine, 100 μg/ml sodium pyruvate, 50 μg/ml 2-Phospho-L-ascorbate trisodium salt, 10% FBS, 50 μg/ml streptomycin, 0.5 μg/ml of Amphotericin B and 0.1 mM L-Homopropargylglycine (HPG). Media was changed every 2 days. After 4 days, the hydrogels were fixed with formalin (30 minutes, room temperature) and then washed with PBS three times (5 minutes, room temperature). Then, the cells were stained with HCS Cell Mask Blue Stain (1:1000, 30 minutes, room temperature) in PBS. The hydrogels were then further washed with 1% BSA (10 min, room temperature) three times. Upon completion of the washes, a monofunctional azide was added in PBS at a dilution of 1:500 (45 min, room temperature). Again, the hydrogels were washed three times with 1% BSA in PBS and then an Alexa Flour 647 azide dye in a copper sulfate solution was added to the hydrogels (overnight, 4° C.). The following day, the hydrogels were washed three times with PBS (10 min, room temperature) and then imaged. The deposited protein was imaged using confocal microscopy (Zeiss LSM 710) and the deposited protein thickness was quantified. Briefly, the maximum intensity projection of a z-slice encompassing a 1 μm section of the cell was projected for each channel and the resulting projection was binarized utilizing Otsu's thresholding. The HCS Cell Mask channel was subtracted from the nascent protein channel to yield a mask illustrating only nascent protein that was secreted on the cell exterior for the given 1 μm section of the cell being analyzed. The thickness of the secreted protein was quantified using the ‘Bond’ plugin (ImageJ) and the mean thickness as well as the maximum thickness were reported for each slice. A total of 5 slices were analyzed per cell, and a total of 30 cells were analyzed per condition to yield significance.

Statistical Analysis. All data was collected using 3 hydrogel replicates per condition. For each hydrogel at least 30 cells were analyzed. With the nascent protein deposition 30 cells were analyzed per hydrogel, with 5 z-slices per cell analyzed. Data was compared using one-way ANOVAs with Tukey post-hoc comparisons or Student's t-test. Data are presented as mean±standard deviation.

All of the COMPOSITIONS and METHODS disclosed and claimed herein can be made and executed without undue experimentation in light of the present disclosure. While the COMPOSITIONS and METHODS have been described in terms of preferred embodiments, it will be apparent to those of skill in the art that variation can be applied to the COMPOSITIONS and METHODS and in the steps or in the sequence of steps of the METHODS described herein without departing from the concept, spirit and scope of the invention. More specifically, it will be apparent that certain agents which are both chemically and physiologically related can be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprising:
a first polymer backbone comprising at least one hyaluronic acid backbone functionalized with an aliphatic aldehyde; and at least one hyaluronic acid backbone functionalized with a hydrazide; and,
a second polymer backbone comprising at least one 8-arm poly(ethylene glycol) (PEG),
wherein the combination of the first and second polymer backbones produces a hybrid network hydrogel.

2. The composition according to claim 1, wherein the at least one 8-arm PEG comprises at least one 8-arm PEG functionalized with at least one strained cyclooctyne.

3. The composition according to claim 2, wherein the at least one strained cyclooctyne comprises a bicyclononyne.

4. The composition according to claim 1, wherein the second polymer backbone comprises at least one 8-arm PEG and further comprising at least one benzaldehyde-PEG3-azide.

5. The composition according to claim 1, wherein the at least one hyaluronic acid backbone, the at least one 8-arm PEG, or a combination thereof is modified with at least one peptide.

6. The composition according to claim 1, wherein the composition comprises in the first polymer backbone, 25% by weight of total crosslink concentration in the polymer backbone comprising at least one hyaluronic acid backbone functionalized with an aliphatic aldehyde and at least one hyaluronic acid backbone functionalized with a hydrazide; and 75% by weight of total crosslink concentration in the second polymer backbone comprising at least one 8-arm PEG.

7. The composition according claim 1, wherein the composition further comprises stem cells.

8. The composition according to claim 7, wherein the stem cells comprise one or more of embryonic stem cells, induced pluripotent stem cells, hematopoietic stem cells, neural stem cells, epithelial stem cells, skin stem cells, and mesenchymal stem cells.

9. The composition according to claim 1, wherein the composition is formulated in a pharmaceutical composition, which further comprises a pharmaceutically acceptable carrier.

10. (canceled)

11. The composition according to claim 1, wherein the composition further comprises at least one active therapeutic agent of use to treat a health condition.

12. The composition according to claim 1, wherein the composition degrades in a subject after at least one month.

13. The composition according to claim 11, wherein the at least one therapeutic-agent comprises a releasable active agent from the composition before, after, or during degradation of the composition after administering the composition to a subject.

14. A method of generating a hybrid network hydrogel according to claim 1, the method comprising combining one polymer backbone comprising at least one hyaluronic acid backbone functionalized with an aliphatic aldehyde and at least one hyaluronic acid backbone functionalized with a hydrazide with a second polymer backbone comprising at least one 8-arm poly(ethylene glycol) (PEG), wherein the method does not require external stimulation for hydrogel formation.

15. The method according to claim 14, wherein the second polymer backbone comprising at least one 8-arm PEG comprises at least one 8-arm PEG functionalized with a bicyclononyne.

16. The method according to claim 14, wherein the second polymer backbone comprising at least one 8-arm PEG further comprises benzaldehyde-PEG3-azide.

17. The method according to claim 14, wherein stress relaxation of the hybrid network hydrogel is increased by combining an increasing concentration of the second polymer backbone comprising at least one 8-arm PEG to a decreasing concentration of the polymer backbone comprising at least one hyaluronic acid backbone functionalized with an aliphatic aldehyde and at least one hyaluronic acid backbone functionalized with a hydrazide.

18. The method according to claim 14, wherein stress relaxation of the hybrid network hydrogel is decreased by combining a decreasing concentration of the second polymer backbone comprising at least one 8-arm PEG to an increasing concentration of the polymer backbone comprising at least one hyaluronic acid backbone functionalized with an aliphatic aldehyde and at least one hyaluronic acid backbone functionalized with a hydrazide.

19. The method according to claim 14, wherein the hybrid network hydrogel further comprises at least one stem cell.

20-23. (canceled)

24. A method of treating, reducing the onset, reducing progression or preventing a health condition in a subject, the method comprising administering to the subject a therapeutically effective amount of a composition according to claim 9.

25-27. (canceled)

28. A kit comprising the according to claim 1, and at least one container.

29. (canceled)
